(19) United States
(12) Patent Application Publication
(10) Pub. No.: US 2016/0375155 A1
(43) Pub. Date: Dec. 29, 2016

(54) Collagen Imaging Compositions

(71) Applicant: Collagen Medical, LLC, Boston, MA (US)

(72) Inventors: Richard L. Looby, Reading, MA (US); Peter D. Caravan, Cambridge, MA (US)

(21) Appl. No.: 14/754,485

(22) Filed: Jun. 29, 2015

(51) Int. Cl. A61K 49/14; C07K 7/54; C07K 7/08

(52) U.S. Cl. CPC A61K 49/14; C07K 7/08; C07K 7/54

(57) ABSTRACT

Compounds and methods for imaging and/or assessing collagen are described. The compounds can include collagen binding peptides.

### FEDERALLY SPONSORED RESEARCH

This invention was made with government support under NIH SBIR grants 5R44DK095617-03 (NIDDK); 4R44HL117488-02 (NHLBI), HHSN268201300054C (NHLBI), and HHSN268201400044C (NHLBI). The government has certain rights in the invention.

### TECHNICAL FIELD

This disclosure relates to compounds that are capable of binding to, and in some cases, imaging collagen, and more particularly to the use of such compounds and pharmaceutical compositions for organ fibrosis imaging, myocardial imaging and perfusion measurements.

### BACKGROUND

Provided herein are improved compounds for binding to and imaging collagen. Also provided herein are pharmaceutical compositions containing the compounds provided herein.

Collagens are a class of extracellular matrix proteins that represent 30% of total body protein and shape the structure of tendons, bones, and connective tissues. Abnormal or excessive accumulation of collagen in organs such as the liver, lungs, kidneys, or breasts, and vasculature can lead to fibrosis of such organs (e.g., myocardial fibrosis, heart failure, nonalcoholic steatohepatitis of the liver (also known as NASH), cirrhosis of the liver, primary biliary cirrhosis), lesions in the vasculature or breasts, collagen-induced arthritis, Muscular dystrophy, scleroderma, Dupuytren's disease, rheumatoid arthritis, and other collagen vascular diseases. It would be useful to have diagnostic agents that could assist in the treatment or diagnosis of such disorders.

Compounds and pharmaceutical compostions for collagen imaging have been previously disclosed in U.S. Pat. No. 8,034,898 and various publications, including Kolodziej et. al., “Peptide optimization and conjugation strategies in the development of molecularly targeted magnetic resonance imaging contrast agents.” Methods Mol Biol. 2014; 1088:185-211, Helm et. Al. “Postinfarction myocardial scarring in mice: molecular magnetic resonance (MR) imaging with use of a collagen-targeting contrast agent.” Radiology. 2008 June; 247(3):788-96, and Caravan et. al. “Collagen-targeted MRI contrast agent for molecular imaging of fibrosis.” Angew Chem Int Ed Engl. 2007; 46(43):8171-3”.

However, improved compounds that exhibit superior binding to collagen of animals used in preclinical studies (especially rodent, canine) along with greater in vivo uptake into collagen tissue and robust imaging enhancement are needed.

### SUMMARY

Provided herein is a compound (Compound ID No. 5) having the following structure:

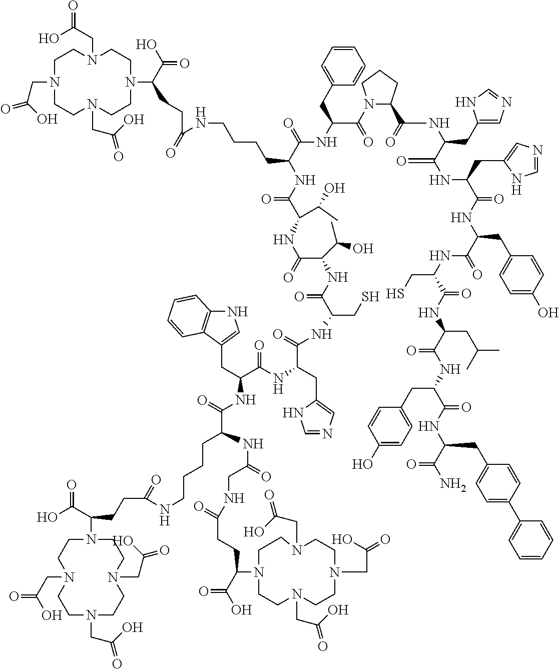

or a pharmaceutically acceptable salt thereof.

In some embodiments, the compound is cyclized through a Cysteine-Cysteine disulfide bond (Compound ID No. 9).

In some of the above embodiments, the compound is complexed to one or more paramagnetic metal ions. For example, the compound can be complexed with one or more of the metal ions selected from the group consisting of: Gd(III), Fe(III), Mn(II), Mn(III), Cr(III), Cu(II), Dy(III), Ho(III), Er(III), Pr(III), Eu(II), Eu(III), Tb(III), and Tb(IV). In some embodiments, the paramagnetic metal ion is Gd(III).

Also provided herein is a compound (Compound ID No. 1) having the following structure:

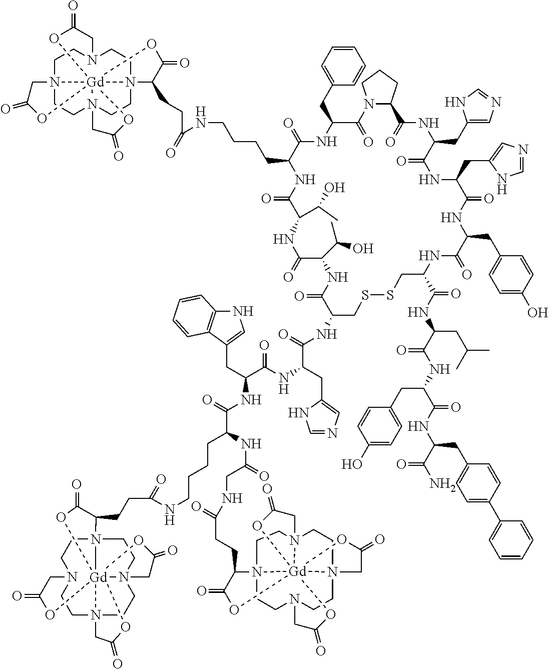

or a pharmaceutically acceptable salt thereof.

In some embodiments, the pharmaceutically acceptable salt is sodium.

The present disclosure also provides methods for using a compound provided herein.

In some embodiments, a method of distinguishing fibrotic from non-fibrotic pathologies in an animal is provided, the method comprising:

administering to the animal an effective amount of an MR composition, the MR composition comprising Compound ID No. 1;

acquiring a T1-weighted image of a tissue of said animal at from about 1 minute to about 10 minutes after administration of the MR composition;

acquiring a second T1-weighted image of the tissue of said animal at a time from about 10 minutes to about 2 hours after administration of the MR composition; and

evaluating differences between the images acquired in steps b) and c), wherein a non-fibrotic tissue exhibits a greater loss in enhancement from the image collected in step b) to that in step c) as compared to a fibrotic pathology.

In some embodiments, a method of distinguishing fibrotic from non-fibrotic pathologies in an animal is provided, the method comprising:

acquiring a T1-weighted image of a tissue of said animal;

administering to the animal an effective amount of an MR composition, the MR composition comprising Compound ID No. 1;

acquiring a T1-weighted image of a tissue of said animal at from about 1 minute to about 60 minutes after administration of the MR composition; and

evaluating differences between the images acquired in steps a) and c), wherein a fibrotic pathology exhibits greater signal increase in the image collected in step c) compared to the image in step a) as compared to non-fibrotic tissue.

Further provided herein is a method of distinguishing fibrotic from non-fibrotic pathologies in an animal, the method comprising:

administering to the animal an effective amount of an MR composition, the MR composition comprising Compound ID No. 1;

measuring R1 (1/T1) of a tissue of said animal at from about 1 minute to about 60 minutes after administration of the MR composition; and

comparing R1 of the tissue to a reference value for that tissue whereby the tissue is fibrotic if the R1 value is greater than the reference value.

Also provided herein is a method of distinguishing fibrotic from non-fibrotic pathologies in an animal, the method comprising:

measuring R1 (1/T1) of a tissue of said animal;

administering to the animal an effective amount of an MR composition, the MR composition comprising Compound ID No. 1;

measuring R1 (1/T1) of a tissue of said animal at from about 1 minute to about 60 minutes after administration of the MR composition; and

comparing the difference in R1 of the tissue before and after administration of the MR composition comprising Compound ID No. 1 (delta-R1) to a reference value for that tissue whereby the tissue is fibrotic if the delta-R1value is greater than the reference value.

In some embodiments, a method of magnetic resonance (MR) imaging for evaluating myocardial perfusion in an animal is provided, the method comprising:

inducing hyperemia in an animal;

administering to the animal an effective amount of an MR composition, the MR composition comprising Compound ID No. 1;

acquiring an MR image of the animal's myocardial tissue after the induction of peak hyperemia in the animal, the acquisition of the MR image beginning at a time frame at least 2 times greater than that required for a first pass distribution of Compound ID No. 1;

acquiring a second MR image of the animal's myocardial tissue after the induction of peak hyperemia in the animal, the acquisition of the MR image beginning at a time frame at least 4 times greater than that required for a first pass distribution of Compound ID No. 1; and

evaluating said images of the animal's myocardial tissue to evaluate myocardial perfusion.

In some embodiments, the method further comprises acquiring an MR image of the myocardial tissue of the animal in a pre-hyperemic state, the MR image in the pre-hyperemic state acquired either before the induction of peak hyperemia in the animal or after a sufficient period of time after the induction of peak hyperemia in the animal to allow the animal to return to a pre-hyperemic state. For example, the evaluating can include comparing the MR images of the myocardial tissue after the induction of peak hyperemia with the MR image of the myocardial tissue in the pre-hyperemic state. In some embodiments, ischemic regions appear hypointense on a T1-weighted image relative to normal, well-perfused myocardial tissue in the image of step c). In some embodiments, non-viable, infarcted tissues appear hyperintense on a T1-weighted image relative to normal, well-perfused myocardial tissue in the image of step d).

Also provided herein is a method of magnetic (MR) imaging for evaluating myocardial perfusion in an animal comprising:

inducing peak hyperemia in an animal;

administering to the animal an effective amount of an MR composition, the MR composition comprising Compound ID No. 1;

acquiring an MR image of the animal's myocardial tissue after the induction of peak hyperemia in the animal, the acquisition of the MR image beginning at a time frame at least 2 times greater than that required for a first pass distribution of Compound ID No. 1; and

evaluating said images of the animal's myocardial tissue to evaluate myocardial perfusion.

In some embodiments, the method further comprises The method according to acquiring an MR image of the myocardial tissue of the animal in a pre-hyperemic state, the MR image in the prehyperemic state acquired either before the induction of peak hyperemia in the animal or after a sufficient period of time after the induction of peak hyperemia in the animal to allow the animal to return to a pre-hyperemic state.

In some embodiments, the method further comprises:

acquiring a second MR image of the animal's myocardial tissue after the induction of peak hyperemia in the animal, the acquisition of the MR image beginning at a time frame at least 4 times greater than that required for a first pass distribution of Compound ID No. 1; and

evaluating the acquired images of the animal's myocardial tissue to evaluate myocardial perfusion.

In some such embodiments, the evaluating includes comparing the MR images of the myocardial tissue after the induction of peak hyperemia with the MR image of the myocardial tissue in the pre-hyperemic state.

In some embodiments, ischemic regions appear hypointense on a T1-weighted image relative to normal, well-perfused myocardial tissue. In some embodiments, non-viable, infarcted tissues appear hyperintense on a T1-weighted image relative to normal, well-perfused myocardial tissue.

### DESCRIPTION OF DRAWINGS

FIG. 1. shows the chemical structure of Compound ID No. 1

FIG. 2. shows the chemical structure of Compound ID No. 2

FIG. 3. shows the chemical structure of Compound ID No. 3

FIG. 4. shows the chemical structure of Compound ID No. 4

FIG. 5. shows the chemical structure of Compound ID No. 5

FIG. 6. shows the chemical structure of Compound ID No. 6

FIG. 7. shows the chemical structure of Compound ID No. 7

FIG. 8. shows the chemical structure of Compound ID No. 8

FIG. 9. shows the chemical structure of Compound ID No. 9

FIG. 10. shows the chemical structure of Compound ID No. 10

FIG. 11. shows the chemical structure of Compound ID No. 11

FIG. 12. shows the chemical structure of Compound ID No. 12

FIG. 13. is a general scheme for preparing Gd-DOTAGA peptide conjugate compounds.

FIG. 14. shows chemical synthesis steps for preparing Compound ID No. 1.

FIG. 15. is the pharmacokinetic profile of Compound ID No. 1 (rat, 1.3 umol/kg iv bolus in 80 mM sucrose, pH 7).

FIG. 16. is a graph of hydroxyproline concentration (collagen) vs. gadolinium concentration (Compound ID. No. 1) for healthy and infarcted heart (n=3 animals).

FIG. 17. is a graph of hydroxyproline concentration (collagen) vs. gadolinium concentration (Compound ID. No. 2) for healthy and infarcted heart (n=3 animals).

FIG. 18. is a graph of hydroxyproline concentration (collagen) vs. gadolinium concentration (Compound ID. No. 4) for healthy and infarcted heart (n=3 animals).

FIG. 19. is a bar graph of the measured slopes for hydroxyproline vs. gadolinium concentration in the rat MI model for the Compound ID Nos. 1, 2, and 4.

FIG. 20. is the study design for Compound ID No. 1 assessment of perfusion in a canine model.

FIG. 21. shows pre- and post-Compound ID No. 1 contrast enhanced images of canine hearts. The perfusion defect is not apparent before Compound ID No. 1 injection. The myocardial perfusion defect (arrow) in the LAD territory is readily apparent after Compound ID No. 1 injection as a hypointense (dark) signal, indicated with arrows, while the normal myocardium is seen with bright signal.

FIG. 22. shows post-Compound ID No. 1 contrast enhanced images of a single canine heart demonstrating the steady-state, whole heart imaging enabled by Compound ID No. 1.

FIG. 23. shows the myocardial signal to noise ratio (SNR) of normally perfused and hypoperfused regions in each animal following administration of Compound ID No. 1.

FIG. 24a and FIG. 24b. Shows the average T1 measurements following administration of Compound ID. No. 1 in an acute myocardial infarction canine heart model (FIG. 24a) and chronic myocardial infarction canine heart model (FIG. 24b). FIG. 24a shows that the T1 of acute infarct is longer than normal (N1) myocardium in acute MI shortly after injection, and myocardium recovers quickly. FIG. 24b shows that chronic scarring shows early and persistent lowering of T1 values, indicating uptake of Compound ID. No. 1 compared to healthy myocardium.

FIG. 25a and FIG. 25b. shows myocardial magnetic resonance imaging following administration of Compound ID. No. 1 in the acute myocardial infarction canine model (FIG. 25a) and chronic myocardial infarction model (FIG. 25b). FIG. 25a shows that imaging of the acute infarction shows lack of contrast early, equilibration with normal myocardium at 30 minutes, and enhancement >1 hr. FIG. 25b shows that chronic scarring shows early uptake of Compound ID. No. 2 compared to healthy myocardium, and persists throughout the imaging session.

FIG. 26. shows a graph of hydroxyproline concentration (collagen) vs. gadolinium concentration (Compound ID No. 1) for healthy, ischemic, and infarcted canine heart tissue (n=2 animals).

### DETAILED DESCRIPTION

### Definitions

Commonly used chemical abbreviations that are not explicitly defined in this disclosure may be found in The American Chemical Society Style Guide, Second Edition; American Chemical Society, Washington, D.C. (1997), “2001 Guidelines for Authors” J. Org. Chem. 66(1), 24A (2001), “A Short Guide to Abbreviations and Their Use in Peptide Science” J. Peptide. Sci. 5, 465-471 (1999).

As used herein, the term “peptide” refers to a chain of amino acids that is 16 or 17 amino acids in length. All peptide sequences herein are written from the N to C terminus. Additionally, the peptides described herein contain two or more cysteine residues that can form one or more disulfide bonds under non-reducing conditions. Formation of a disulfide bond can result in the formation of a cyclic peptide.

As used herein, the term “natural” or “naturally occurring” amino acid refers to one of the twenty most common occurring amino acids. Natural amino acids modified to provide a label for detection purposes (e.g., radioactive labels, optical labels, or dyes) are considered to be natural amino acids. Natural L amino acids are referred to by their standard one- or three-letter abbreviations.

For the purposes of this application, “DTPA derivative” refers to a chemical compound comprising a substructure composed of diethylenetriamine, wherein the primary and secondary amines are each covalently derivatized according to the following formula:

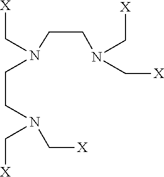

wherein each X is independently a functional group capable of coordinating a metal cation, preferably selected from the group consisting of COOR, C(O)NRR′, PO₃RR′⁻, P(R)O₂R′, NRR′, and OR, wherein R and R′ are independently selected from hydrogen, methyl, ethyl, propyl isopropyl, butyl, isobutyl, tert-butyl or other C1 to C6 aliphatic moiety, which can be saturated, unsaturated, cyclic, branched, or straight chain. It is assumed that a person of ordinary skill would understand that, depending on the pH of the medium, certain moieties may be charged or uncharged. Similarly, a person having ordinary skill in the art would understand that the structures can coordinate appropriately charged metal ions. When each X group is the carboxyl moiety (COOH) or carboxylate (COO⁻), then the structure may be referred to as “DTPA”. When each X group is the tert-butoxy (^{t}Bu) carboxylate ester (COO^{t}Bu), the structure may be referred to as “DTPE” (“E” for ester). When each X group is the carboxylate (COO⁻) or carboxyl moiety and coordinated to gadolinium(III), the structure may be referred to as “GdDTPA” and includes pharmaceutically acceptable salts thereof. It is understood by persons familiar with the art that an exchangeable water molecule (H₂O) may also be coordinated to any such coordinated metal ion. For example, an exchangeable water molecule is typically coordinated to gadolinium in GdDTPA as well as the nitrogen and oxygen atoms of the DTPA chelating ligand.

For the purposes of this application, “DOTA” refers to a chemical compound comprising a substructure composed of 1,4,7,11-tetraazacyclododecane, wherein the four secondary amines are each covalently derivatized according to the following formula:

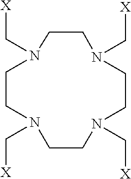

wherein X is defined above. It is assumed that a person of ordinary skill would understand that, depending on the pH of the medium, certain moieties may be charged or uncharged. Similarly, a person having ordinary skill in the art would understand that the structures can coordinate appropriately charged metal ions. When each X group is the carboxylate (COO⁻) and coordinated to gadolinium(III), the structure may be referred to as “GdDOTA” and includes pharmaceutically acceptable salts thereof. It is understood by persons familiar with the art that an exchangeable water molecule (H₂O) may also be coordinated to any such coordinated metal ion. For example, an exchangeable water molecule is typically coordinated to gadolinium in GdDOTA as well as the nitrogen and oxygen atoms of the DOTA chelating ligand.

For the purposes of this application, “NOTA” refers to a chemical compound comprising a substructure composed of 1,4,7-triazacyclononane, wherein the secondary amines are each covalently derivatized according to the following formula:

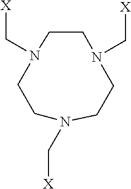

wherein X is defined above. It is assumed that a person of ordinary skill would understand that, depending on the pH of the medium, certain moieties may be charged or uncharged. Similarly, a person having ordinary skill in the art would understand that the structures can coordinate appropriately charged metal ions. It is understood by persons familiar with the art that an exchangeable water molecule (H₂O) may also be coordinated to any such coordinated metal ion.

For the purposes of this application, “DOTAGA derivative” refers to a chemical compound comprising a substructure composed of 1,4,7,11-tetraazacyclododecane, wherein the primary and secondary amines are each covalently derivatized according to the following formula,

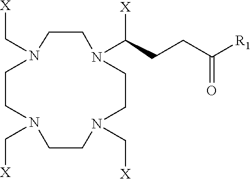

wherein X is defined above and R¹═OH, O-tBu, or NRR′ where R and R′ are independently selected from hydrogen, a peptide, methyl, ethyl, propyl isopropyl, butyl, isobutyl, tert-butyl or other C1 to C6 aliphatic moiety, which can be saturated, unsaturated, cyclic, branched, or straight chain. When each X group is the carboxyl moiety (COOH) or carboxylate moiety (COO), then the structure may be referred to as “DOTAGA” and includes pharmaceutically acceptable salts thereof. When each X group is the tert-butoxy (^{t}Bu) carboxylate ester (COO^{t}Bu), the structure may be referred to as “DOTAGA(O^{t}Bu)₄”. It is assumed that a person of ordinary skill would understand that, depending on the pH of the medium, certain moieties may be charged or uncharged. Similarly, a person having ordinary skill in the art would understand that the structures can coordinate appropriately charged metal ions. When each X group is the carboxylate (COO⁻) and coordinated to gadolinium(III), the structure may be referred to as “GdDOTAGA” and includes pharmaceutically acceptable salts thereof. It is understood by persons familiar with the art that an exchangeable water molecule (H₂O) may also be coordinated to any such coordinated metal ion. For example, an exchangeable water molecule is typically coordinated to gadolinium in GdDOTAGA as well as the nitrogen and oxygen atoms of the DOTAGA chelating ligand.

For the purposes of this application, “DO3A” refers to a chemical compound comprising a substructure composed of 1,4,7,11-tetraazacyclododecane, wherein three of the four amines are each covalently derivatized according to the following formula and the other amine has a substituent having neutral charge according to the following formula:

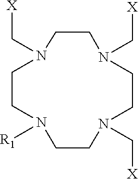

wherein each X is independently a functional group capable of coordinating a metal cation, preferably selected from the group consisting of COOR, C(O)NRR′, PO₃RR′⁻, P(R)O₂R′, NRR′, and OR, wherein R and R′ are independently selected from hydrogen, methyl, ethyl, propyl isopropyl, butyl, isobutyl, tert-butyl or other C1 to C6 aliphatic moiety, which can be saturated, unsaturated, cyclic, branched, or straight chain. It is assumed that a person of ordinary skill would understand that, depending on the pH of the medium, certain moieties may be charged or uncharged. Similarly, a person having ordinary skill in the art would understand that the structures can coordinate appropriately charged metal ions. When each X group is the carboxyl moiety (COOH) or carboxylate moiety (COO⁻) and pharmaceutically acceptable salts thereof and R₁═H, then the chelating moiety is “DO3A”. When each X group is the carboxyl moiety (COOH) or carboxylate moiety (COO⁻) and R₁═—CH₂(CHOH)CH₃, then the chelating moiety is “HP-DO3A”. It is understood by persons familiar with the art that an exchangeable water molecule (H₂O) may also be coordinated to any such coordinated metal ion. For example, an exchangeable water molecule is typically coordinated to gadolinium in GdDO3A as well as the nitrogen and oxygen atoms of the DO3A chelating ligand.

In each of the four structures above, the carbon atoms of the indicated ethylenes may be referred to as “backbone” carbons. The designation “bbDTPA” may be used to refer to the location of a chemical bond to a DTPA molecule (“bb” for “back bone”). Note that as used herein, Gd(bb(CO)DTPA) means a C═O moiety bound to an ethylene backbone carbon atom of DTPA.

The terms “chelating ligand,” and “chelating moiety,” may be used to refer to any polydentate ligand which is capable of coordinating a metal ion, including DTPA (and DTPE), DOTA, DO3A, DOTAGA, Glu-DTPA, or NOTA as described above, or derivatives thereof, or any other suitable polydentate chelating ligand as is further defined herein, that is either coordinating a metal ion or is capable of doing so, either directly or after removal of protecting groups. The term “chelate” refers to the actual metal-ligand complex, and it is understood that a polydentate ligand can eventually be coordinated to metal ion, which can be a medically useful metal ion.

The terms “target binding” and “binding” for purposes herein refer to non-covalent interactions of a peptide or composition with a target. These non-covalent interactions are independent from one another and may be, inter alia, hydrophobic, hydrophilic, dipole-dipole, pi-stacking, hydrogen bonding, electrostatic associations, or Lewis acid-base interactions. The binding affinity for a target is expressed in terms of the equilibrium dissociation constant “Kd” to the target under a defined set of conditions.

The term “relaxivity” as used herein, refers to the increase in either of the magnetic resonance imaging (MRI) quantities 1/T1 or 1/T2 per millimolar (mM) concentration of paramagnetic ion, contrast agent, or compound, wherein T1 is the longitudinal or spin-lattice, relaxation time, and T2 is the transverse or spin-spin relaxation time of water protons or other imaging or spectroscopic nuclei, including protons found in molecules other than water. Relaxivity is expressed in units of mM⁻¹s⁻¹.

As used herein, the term “purified” refers to a peptide or compound that has been separated from either naturally occurring organic molecules with which it normally associates or, for a chemically-synthesized molecule, separated from other organic molecules present in the chemical synthesis. Typically, the polypeptide or compound is considered “purified” when it is at least 70% (e.g., 70%, 80%, 90%, 95%, or 99%), by dry weight, free from any other proteins or organic molecules. The terms “purified” and “isolated” are used interchangeably herein.

As used herein, all references to “Gd” or “gadolinium” mean the Gd(III) paramagnetic metal ion.

### Collagen Binding Imaging Compounds

Compounds of the invention (e.g., compounds suitable for MR imaging, optical imaging, and nuclear imaging, including PET imaging and SPECT imaging), which can be used for imaging collagen and for detecting pathologies where abnormal or excessive proliferation of collagen is implicated, are described herein. Compounds of the invention include a collagen binding peptide linked to one or more chelating moieties, which in turn may be coordinated to one or more metal ions.

Collagen Binding Peptides

Compounds described herein have an affinity for the extracellular matrix protein collagen, including human and other animal Collagen Type I. Collagens are particularly useful extracellular matrix proteins to target. For example, collagens I and III are the most abundant components of the extracellular matrix of myocardial tissue, representing over 90% of total myocardial collagen and about 5% of dry myocardial weight. The ratio of collagen I to collagen III in the myocardium is approximately 2:1, and their total concentration is approximately 100 μM in the extracellular matrix. Human collagen type I is a trimer of two chains with an [α1 (I)]₂ [α2(I)] stoichiometry characterized by a repeating G-X-Y sequence motif, where X is most frequently proline and Y is frequently hydroxyproline. In some embodiments, a compound described herein can have an affinity for human, rat, and/or dog collagen type I.

The compounds described herein comprise a collagen binding peptide linked to one or more chelating moieties. Peptides useful for inclusion in the compounds and compositions described herein include natural amino acids and the unnatural amino acid L-4,4′-biphenylalanine (Bip). The peptides can be synthesized according to standard synthesis methods such as those disclosed in, e.g., WO 01/09188 and WO 01/08712. Amino acids with many different protecting groups appropriate for immediate use in the solid phase synthesis of peptides are commercially available.

Peptides can be assayed for affinity to the appropriate extracellular matrix protein by methods as disclosed in WO 01/09188 and WO 01/08712, and as described below. For example, peptides can be screened for binding to an extracellular matrix protein by methods well known in the art, including pull-down assays, equilibrium dialysis, affinity chromatography, and inhibition or displacement of probes bound to the matrix protein. For example, peptides can be evaluated for their ability to bind to collagen, such as dried human, rat or dog collagen type I. In some embodiments a collagen binding peptide can bind human collagen with a dissociation constant of less than 25 μM, less than 10 μM, less than 5 μM, less than 1 μM, or less than 100 nM. In some embodiments the collagen binding peptide can bind rat collagen with a dissociation constant of less than 25 μM, less than 10 μM, less than 5 μM, less than 1 μM, or less than 100 nM. In some embodiments the collagen binding peptide can bind dog collagen with a dissociation constant of less than 25 μM, less than 10 μM, less than 5 μM, less than 1 μM, or less than 100 nM.

A purified peptide of the invention includes one of the following amino acid sequences disclosed herein:

(SEQ ID No. 1)
G-K-W-H-C-T-T-K-F-P-H-H-Y-C-L-Y-Bip;
(SEQ ID No. 2)
K-W-H-C-T-T-K-F-P-H-H-Y-C-L-Y-Bip;
(SEQ ID No. 3)
K-Y-W-H-C-T-T-K-F-P-H-H-Y-C-L-Y-Bip;
or
(SEQ ID No. 4)
K-W-H-C-Y-T-K-F-P-H-H-Y-C-V-Y-Bip,

wherein Bip is

L-4,4′-biphenylalanine.

In a specific embodiment, such a purified peptide includes the amino acid sequence:

(SEQ ID No. 1)
G-K-W-H-C-T-T-K-F-P-H-H-Y-C-L-Y-Bip.

In some embodiments, such a purified peptide includes the amino acid sequence:

(SEQ ID No. 2)
K-W-H-C-T-T-K-F-P-H-H-Y-C-L-Y-Bip.

In some embodiments, such a purified peptide includes the amino acid sequence:

(SEQ ID No. 3)
K-Y-W-H-C-T-T-K-F-P-H-H-Y-C-L-Y-Bip.

In some embodiments, such a purified peptide includes the amino acid sequence:

(SEQ ID No. 4)
K-W-H-C-Y-T-K-F-P-H-H-Y-C-V-Y-Bip.

A purified peptide can include any of the amino acid sequences above, also set forth in Table 1, where the peptide has a total length of 16 or 17 amino acids.

TABLE 1
Collagen binding peptides of the invention.
Depending on the reducing conditions in the
medium, a peptide described herein may be
in a linear or cyclic form or a mixture
thereof; similarly in a composition
comprising a peptide described herein, the
peptide may be present as the linear,
the cyclic, or a mixture of the
cyclic and linear forms.

| | SEQUENCE | | | | | | | | | | | | | | | | |
|---|---|---|---|---|---|---|---|---|---|---|---|---|---|---|---|---|---|
| SEQ | (AA #) | | | | | | | | | | | | | | | | |
| ID | 1 | 2 | 3 | 4 | 5 | 6 | 7 | 8 | 9 | 10 | 11 | 12 | 13 | 14 | 15 | 16 | 17 |
| 1 | G | K | W | H | C | T | T | K | F | P | H | H | Y | C | L | Y | Bip |
| 2 | K | W | H | C | T | T | K | F | P | H | H | Y | C | L | Y | Bip | |
| 3 | K | Y | W | H | C | T | T | K | F | P | H | H | Y | C | L | Y | Bip |
| 4 | K | W | H | C | Y | T | K | F | P | H | H | Y | C | V | Y | Bip | |

### Chelating Moieties and Chelates

A chelating moiety can be any of the many known in the art, for example, cyclic and acyclic organic chelating moieties such as DTPA, DOTA, HP-DO3A, DOTAGA, NOTA, Glu-DTPA, and DTPA-BMA, as described above. The term “chelate” refers to a metal-ligand complex.

For magnetic resonance imaging agents, a paramagnetic metal ion such as Gd(III), Fe(III), Mn(II), Mn(III), Cr(III), Cu(II), Dy(III), Ho(III), Er(III), Pr(III), Eu(II), Eu(III), Tb(III), and Tb(IV) can be particularly useful to coordinate to a chelating moiety, and can be complexed to the chelating moieties as previously described. It is understood by persons familiar with the art that an exchangeable water molecule (H₂O) may also be coordinated to the paramagnetic metal as part of the chelate. For example, an exchangeable water molecule is typically coordinated to gadolinium in GdDOTAGA as well as the nitrogen and oxygen atoms of the DOTAGA chelating ligand.

For MRI, metal chelates such as gadolinium diethylenetriaminepentaacetate (GdDTPA), gadolinium tetraamine 1,4,7,10-tetraazacyclododecane-N,N′,N″,N′″-tetraacetate (GdDOTA), gadolinium 1,4,7,10-tetraazacyclododecane-1,4,7-triacetate (GdDO3A), and Gd(bb(CO)DTPA) are particularly useful. In certain embodiments, macrocylic chelating moieties such as DOTAGA are preferred. When complexed to gadolinium(III), the resulting structure may be referred to as “GdDOTAGA” and includes pharmaceutically acceptable salts thereof. The structure of DOTAGA complexed with Gd(III) and having a carboxyl side chain is as follows:

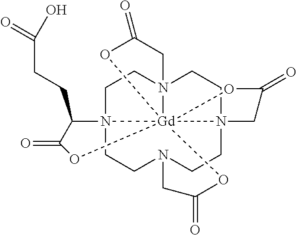

or pharmaceutically acceptable salts thereof. Persons familiar with the art understand that an exchangeable water molecule (H₂O) is typically coordinated to gadolinium as well as the nitrogen and oxygen atoms of the DOTAGA chelating ligand.

For radionuclide imaging agents, radionuclides ⁶⁰Cu, ⁶¹Cu, ⁶²Cu, ⁶⁴Cu, ⁶⁸Ga, ⁹⁴Tc, ⁸⁶Y, ⁸⁹Zr, ⁵¹Mn, ⁵²Mn, ⁴⁴Sc, Al, ¹⁸F, ⁹⁰Y, ^{99m}Tc, ¹¹¹In, ⁴⁷C, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb, and ¹⁴¹Ce are particularly useful, and can be complexed to the chelating moieties described previously.

Metal complexes with useful optical properties also have been described. See, Murru et al., J. Chem. Soc. Chem. Comm. 1993, 1116-1118. For optical imaging using chelates, lanthanide chelates such as La(III), Ce(III), Pr(III), Nd(III), Pn(III), Sm(III), Eu(III), Gd(III), Tb(III), Dy(III), Ho(III), Er(III), Tm(III), Yb(III) and Ln(III) are suitable. Eu(III) and Tb(III) are particularly useful.

Metal chelates should not dissociate metal to any significant degree during the imaging agent's passage through the body, including while bound to a target tissue.

Compounds of the invention are synthesized using literature methods described in U.S. Pat. No. 6,991,775, U.S. Pat. No. 8,034,898, and elsewhere, such as in Kolodziej et. al. in Andrew E. Nixon (ed.), Therapeutic Peptides: Methods and Protocols, Methods in Molecular Biology, vol. 1088), and as described herein.

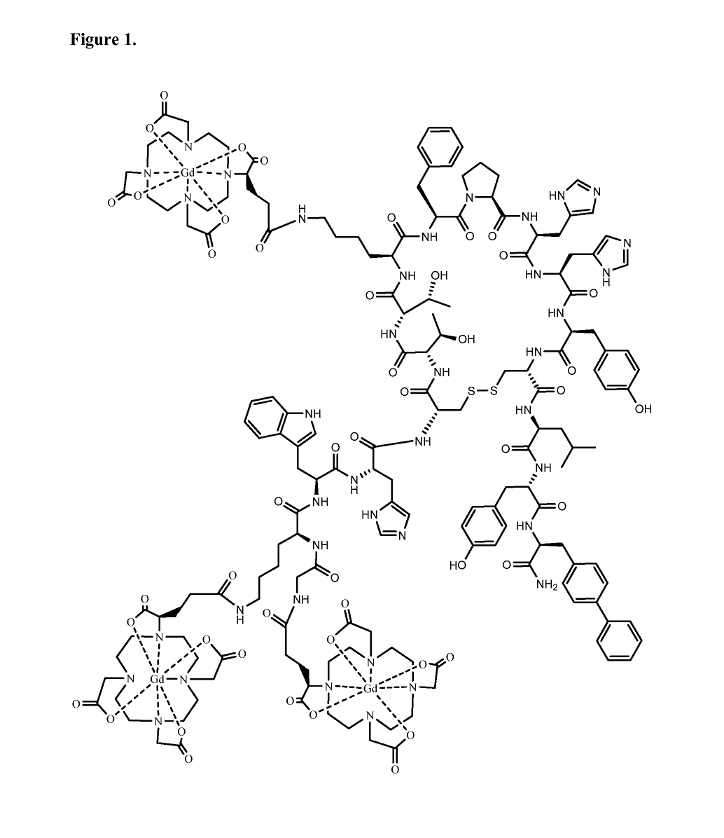

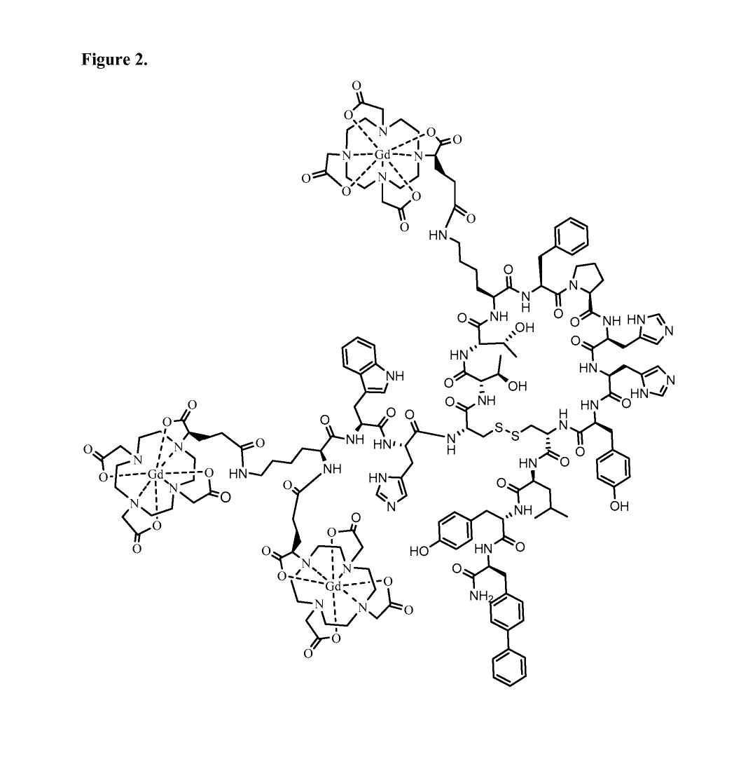

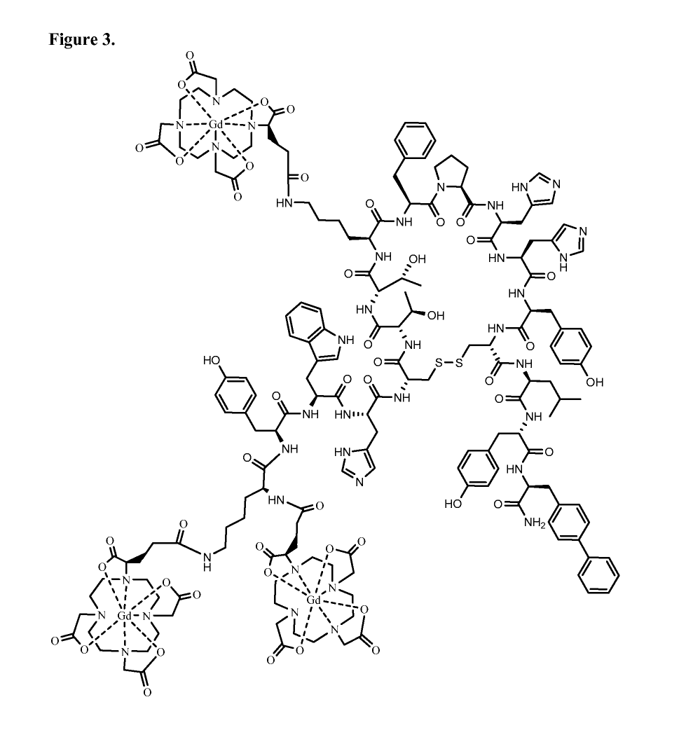

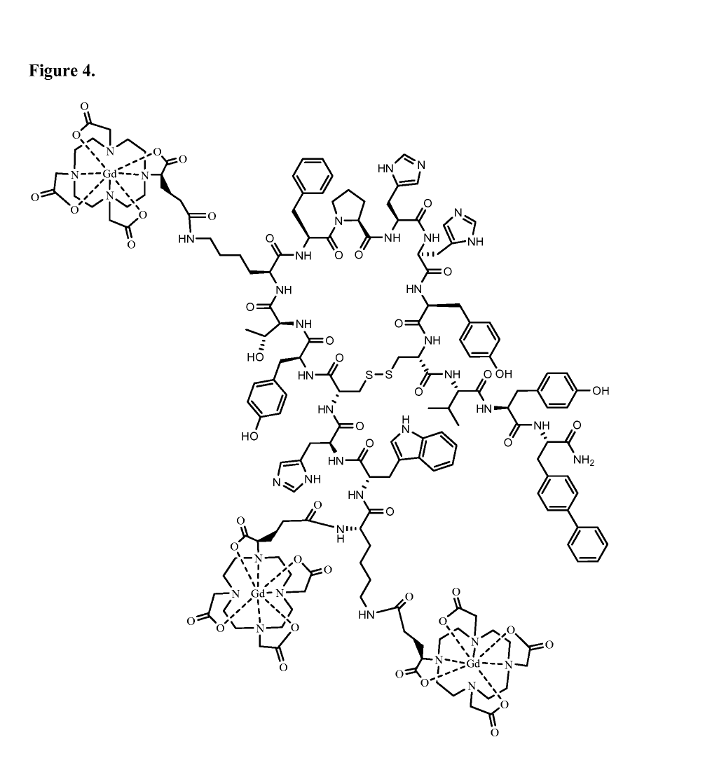

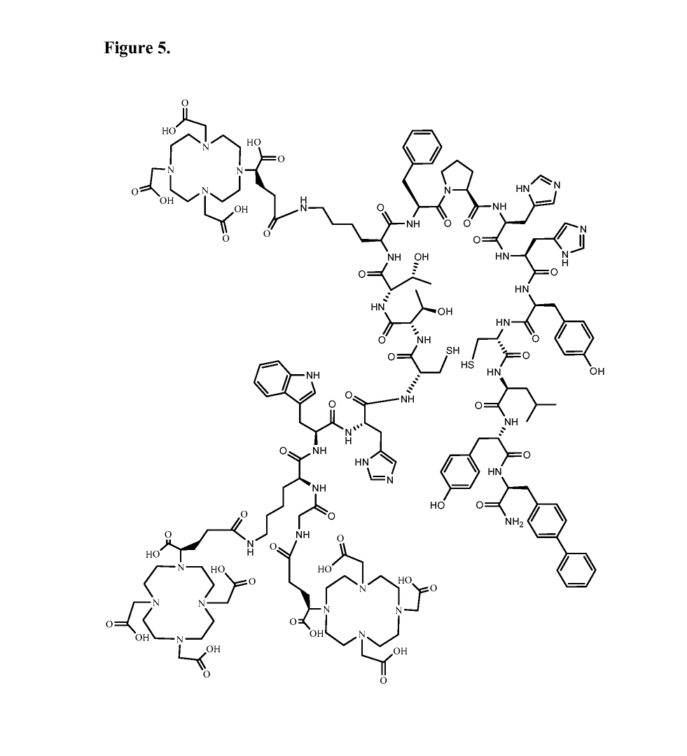

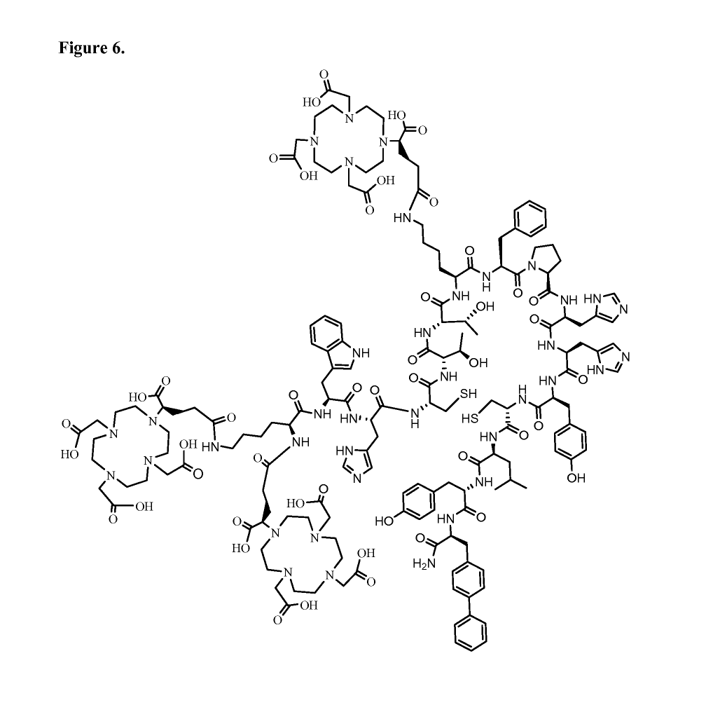

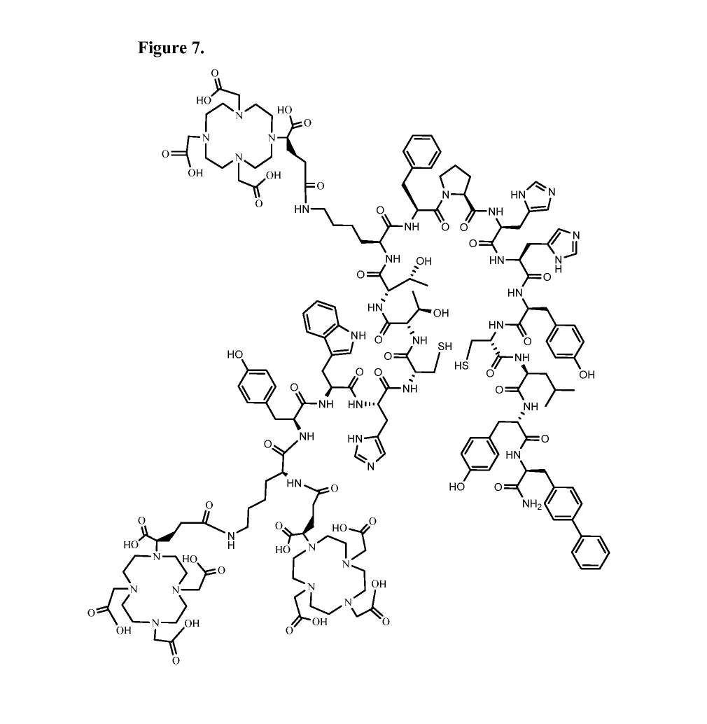

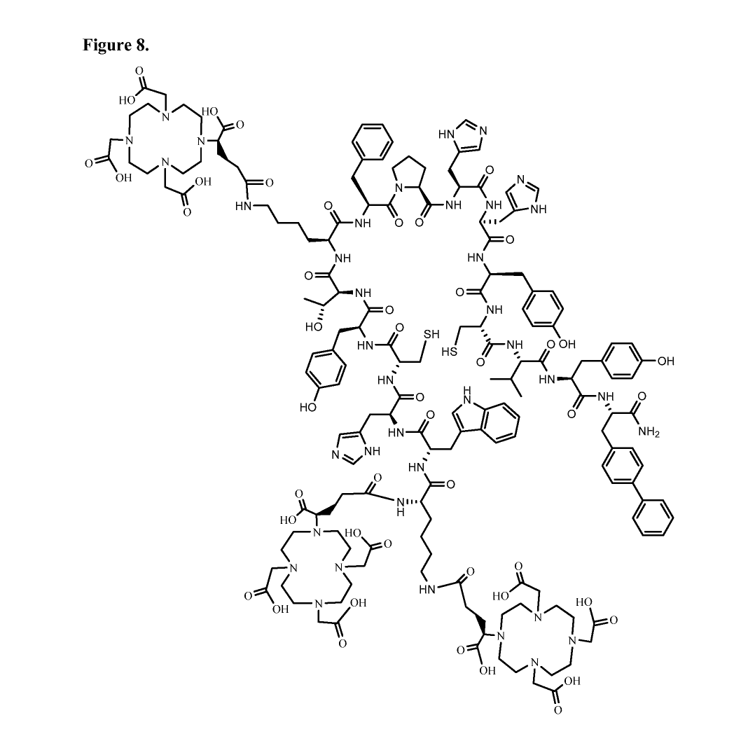

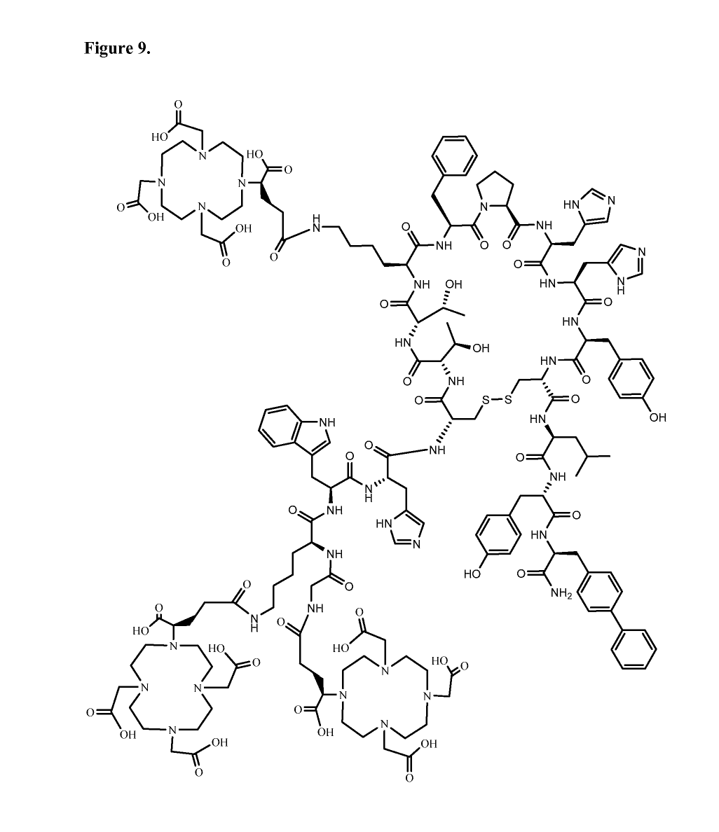

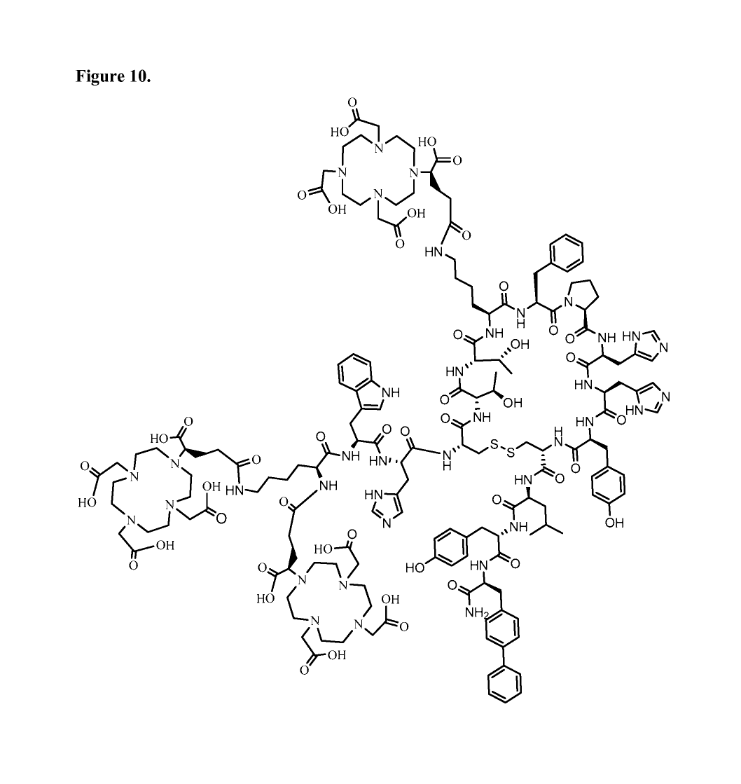

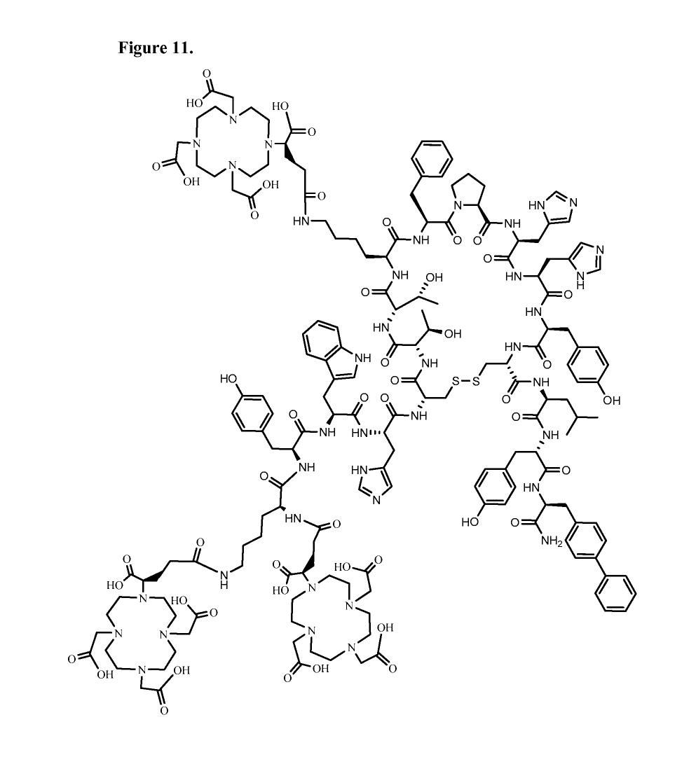

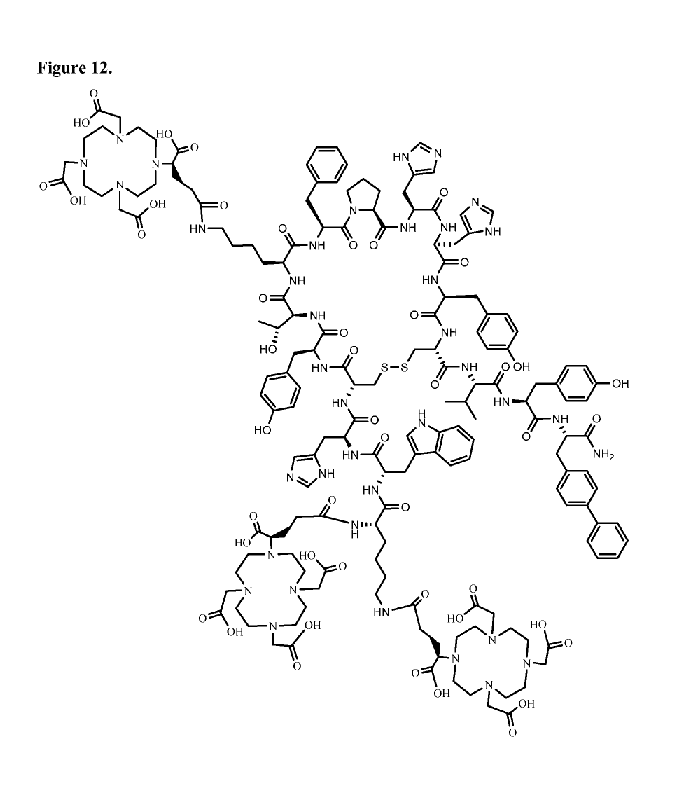

The chemical structures of certain compounds of the invention disclosed herein are shown in FIGS. 1-12.

### Properties of Compounds

Compounds of the invention, including peptides, peptides conjugated chelates, can be formulated as a pharmaceutical composition in accordance with routine procedures. As used herein, the compounds of the invention can include pharmaceutically acceptable derivatives thereof “Pharmaceutically acceptable” means that the compound or composition can be administered to an animal without unacceptable adverse effects. A “pharmaceutically acceptable derivative” means any pharmaceutically acceptable salt, ester, salt of an ester, or other derivative of a compound of this invention that, upon administration to a recipient, is capable of providing (directly or indirectly) a compound of this invention or an active metabolite or residue thereof.

Other derivatives are those that increase the bioavailability of the compounds of this invention when such compounds are administered to a animal (e.g., by allowing an orally administered compound to be more readily absorbed into the blood) or which enhance delivery of the parent compound to a biological compartment (e.g., the brain or lymphatic system) thereby increasing the exposure relative to the parent species.

Pharmaceutically acceptable salts of the compounds of this invention include counter ions derived from pharmaceutically acceptable inorganic and organic acids and bases known in the art. Pharmaceutical compositions of the invention can be administered by any route, including both oral and parenteral administration. Parenteral administration includes, but is not limited to, subcutaneous, intravenous, intraarterial, interstitial, intrathecal, and intracavity administration. When administration is intravenous, pharmaceutical compositions may be given as a bolus, as two or more closes separated in time, or as a constant or non-linear flow infusion. Thus, compositions of the invention can be formulated for any route of administration.

Typically, compositions for intravenous administration are solutions in sterile isotonic aqueous buffer. Where necessary, the composition may also include a solubilizing agent, a stabilizing agent, and a local anesthetic such as lidocaine to ease pain at the site of the injection. The composition for intravenous administration may include 80 millimolar sucrose. Generally, the ingredients will be supplied either separately, e.g. in a kit, or mixed together in a unit dosage form, for example, as a dry lyophilized powder or water free concentrate. The composition may be stored in a hermetically sealed container such as an ampule or sachette indicating the quantity of active agent in activity units. Where the composition is administered by infusion, it can be dispensed with an 10 infusion bottle containing sterile pharmaceutical grade “water for injection,” saline, or other suitable intravenous fluids. Where the composition is to be administered by injection, an ampule of sterile water for injection or saline may be provided so that the ingredients may be mixed prior 15 to administration. Pharmaceutical compositions of this invention comprise the compounds of the present invention and pharmaceutically acceptable salts thereof, with any pharmaceutically acceptable ingredient, excipient, carrier, adjuvant or vehicle.

A compound is preferably administered to the patient in the form of an injectable composition. The method of administering a compound is preferably parenterally, meaning intravenously, intra-arterially, intrathecally, interstitially or intracavitarilly. Pharmaceutical compositions of this invention can be administered to animals including humans in a manner similar to other diagnostic or therapeutic agents. The dosage to be administered, and the mode of administration will depend on a variety of factors including age, weight, sex, condition of the patient and genetic factors, and will ultimately be decided by medical personnel subsequent to experimental determinations of varying dosage followed by imaging as described herein. In general, dosage required for diagnostic sensitivity will range from about 0.001 to 1000 μg/kg, preferably between 0.001 to 25.0 ug/kg of host body mass. The optimal dose will be determined empirically following the disclosure herein.

Compounds of the present invention incorporate the collagen binding peptide sequences described above and physiologically compatible chelating moieties. The compounds thus target extracellular matrix collagen (“the target”), e.g., such as collagen present in the extracellular matrix of the myocardium or liver, and bind to it, allowing imaging of collagen and/or the myocardium or liver.

The extent of binding of a compound to a target can be assessed by a variety of equilibrium binding methods, e.g., ultrafiltration methods; equilibrium dialysis; affinity chromatography; or competitive binding inhibition or displacement of probe compounds. In some cases, peptides can be evaluated for their ability to bind to collagen using assays described herein or as indicated in the cross-referenced application, such as dried human, rat or dog collagen assays. For example, in certain cases, a compound of the invention can bind dried human collagen or dried rat collagen with a dissociation constant of less than 25 μM (e.g., less than 20 μM, less than 10 μM, less than 5 μM, less than 1 μM, or less than 100 nM).

MR compounds can exhibit high relaxivity as a result of binding to collagen, which can lead to better image resolution. The increase in relaxivity upon binding is typically 1.5-fold or more (e.g., at least a 2, 3, 4, 5, 6, 7, 8, 9, or 10 fold increase in relaxivity). Targeted MR compounds having 7-8 fold, 9-10 fold, or even greater than 10 fold increases in relaxivity are particularly useful. Typically, relaxivity is measured using an NMR spectrometer. The preferred relaxivity of an MRI compound at 20 MHz and 37° C. is at least 8 mM⁻¹s⁻¹ per paramagnetic metal ion (e.g., at least 10, 15, 20, 25, 30, 35, 40, or 60 mM⁻¹s⁻¹ per paramagnetic metal ion). MR compounds having a relaxivity greater than 60 mM⁻¹s⁻¹ at 20 MHz and 37° C. are particularly useful.

As described herein, targeted MR compounds can be taken up selectively by areas in the body having higher concentrations of collagen relative to other areas. Selectivity of uptake of targeted agents can be determined by comparing the uptake of the agent by myocardium as compared to the uptake by blood. The selectivity of targeted compounds also can be demonstrated using MRI and observing enhancement of myocardial signal as compared to blood signal.

A compound of the invention may include a variety of physiologically compatible salt forms, including alkali and alkaline earth metal cations, notably sodium. Additional examples include but are not limited to primary, secondary and tertiary amines such as ethanolamine, diethanolamine, morpholine, glucamine, N,N-dimethylglucamine, N-methylglucamine, and amino acids such as lysine, arginine and ornithine.

### Use of Compounds

MR compounds prepared according to the disclosure herein may be used in the same manner as conventional MR compounds and are useful for imaging extracellular matrix collagen, including the myocardium and also fibrotic organ tissue which is rich in Collagen Type 1. Typically, the a composition comprising the MR compound (an MR composition) is administered to a patient (e.g., an animal, such as a human) and an MR image of the patient is acquired. Generally, the clinician will acquire an image of an area having the extracellular matrix component that is targeted by the agent. For example, the clinician may acquire an image of the heart, a joint, a bone, or an organ (e.g., liver, lung, kidney, heart) if the compound targets collagen or locations of abnormal collagen accumulation in a disease state. The clinician may acquire one or more images at a time before, during, or after administration of the MR compound.

Certain MR techniques and pulse sequences may be preferred in the methods of the present disclosure. Both 2-dimensional and 3-dimensional T1-weighted acquisitions are desirable. For example spin-echo and fast spin echo sequences with short repetition times (TR), or gradient recalled echo sequences with short TR. Inversion recovery sequences may be particularly useful for highlighting T1 changes, as well as the use of an inversion prepulse combined with a T1-weighted sequence. For cardiac imaging methods of cardiac gating, either prospective or retrospective methods, can be applied to freeze cardiac motion. Similarly artifacts from respiratory motion can be reduced using breath-hold methodologies or free-breathing navigator techniques. In some instances it may be desirable to obtain additional contrast and the T1-weighted sequence can be combined with fat suppression, or blood flow suppression, or by using a magnetization transfer prepulse. Similarly, those of skill in the art will recognize other suitable MR-based methods for detecting infarct, e.g., T2 weighted imaging, delayed hyperenhancement imaging following extracellular contrast agent, and myocardial imaging.

In some embodiments, fibrotic pathologies are distinguished from non-fibrotic pathologies using a method comprising (a) administering to the animal an effective amount of an MR composition comprising Compound ID No. 1, 2, 3 or 4; (b) acquiring a T1-weighted image of a tissue of said animal at from about 1 minute to about 10 minutes after administration of the MR composition; (c) acquiring a second T1-weighted image of the tissue of said animal at a time from about 10 minutes to about 2 hours after administration of the MR composition; and evaluating differences between the images acquired in steps (b) and (c), wherein a non-fibrotic tissue exhibits greater loss in enhancement from the image collected in step (b) to that in step (c) as compared to a fibrotic pathology.

In another embodiment, a method of distinguishing fibrotic from non-fibrotic pathologies in an animal comprises (a) administering to the animal an effective amount of an MR composition, the MR composition comprising Compound ID No. 1, 2, 3 or 4; (b) measuring R1 (1/T1) of a tissue of said animal at from about 1 minute to about 60 minutes after administration of the composition; and (c) comparing R1 of the tissue to a reference value for that tissue whereby the tissue is fibrotic if the R1 value is greater than the reference value.

In a further embodiment, a method of distinguishing fibrotic from non-fibrotic pathologies in an animal comprises: (a) measuring R1 (1/T1) of a tissue of said animal; (b) administering to the animal an effective amount of an MR composition, the MR composition comprising Compound ID No. 1, 2, 3 or 4; (c) measuring R1 (1/T1) of a tissue of said animal at from about 1 minute to about 60 minutes after administration of the composition; and (d) comparing the difference in R1 of the tissue before and after administration of an MR composition, the MR composition comprising Compound ID No. 1, 2, 3 or 4 (delta-R1) to a reference value for that tissue whereby the tissue is fibrotic if the delta-R1value is greater than the reference value.

In some embodiments, a contrast-enhancing imaging sequence that preferentially increases a contrast ratio of a magnetic resonance signal of tissue, such as the myocardium, having a MR compound bound thereto relative to the magnetic resonance signal of background or flowing blood is used. These techniques include, but are not limited to, black blood angiography sequences that seek to make blood dark, such as fast spin echo sequences; flow-spoiled gradient echo sequences; and out-of-volume suppression techniques to suppress in-flowing blood. These methods also include flow independent techniques that enhance the difference in contrast due to the T1 difference between contrast-enhanced myocardium and blood and tissue, such as inversion-recovery prepared or saturation-recovery prepared sequences that will increase the contrast between the myocardium and background tissues. Methods of preparation for T2 techniques may also prove useful. Finally, preparations for magnetization transfer techniques may also improve contrast with MR compounds.

Methods may be used that involve the acquisition and/or comparison of contrast-enhanced and non-contrast images and/or the use of one or more additional MR appropriate compounds, which may be referred to herein as MR compounds. The additional MR compounds may also exhibit affinity for an extracellular matrix component of the myocardium, as described herein. For example, a series of images may be obtained with an MR compound that binds to collagen, while another series of images may be obtained with an MR compound that binds to elastin. Alternatively, an additional MR compound may be used that is nonspecific or that may exhibit an affinity for fibrin or HSA. For example, methods as set forth in U.S. patent application Ser. No. 09/778,585, entitled MAGNETIC RESONANCE ANGIOGRAPHY DATA, filed Feb. 7, 2001 and U.S. patent application Ser. No. 10/209,416, entitled SYSTEMS AND METHODS FOR TARGETED MAGNETIC RESONANCE IMAGING OF THE VASCULAR SYSTEM, filed Jul. 30, 2002 may be used. Similarly, fibrin targeted agents are described in U.S. patent application Ser. No. 10/209,183, entitled PEPTIDE-BASED MULTIMERIC TARGETED CONTRAST AGENTS, filed Jul. 30, 2002. Compounds for binding HSA are described in WO 96/23526.

In addition, MR compounds are useful for monitoring and measuring myocardial perfusion. Certain methods include the step of obtaining an MR image of the myocardial tissue of an animal while the animal is in a pre-hyperemic state. As used herein, the term “pre-hyperemic state” refers to a resting physiologic state of the animal. In some methods, peak hyperemia can be induced in the animal, either before or after the step of obtaining a pre-hyperemic MR image. As used herein, the term “peak hyperemia” means the point approaching maximum increased blood supply to an organ or blood vessel for physiologic reasons. Peak hyperemia can be exercise-induced or pharmacologically-induced. Exercise-induced peak hyperemia can be achieved through what is commonly known as a “stress test,” and has several clinically relevant endpoints, including excessive fatigue, dyspnea, moderate to severe angina, hypotension, diagnostic ST depression, or significant arrhythmia. If exercise is used to induce peak hyperemia, the animal can exercise for at least one additional minute before the administration of a compound, as described below. The cardiac effect of exercise-induced peak hyperemia can also be simulated pharmacologically (e.g., by the intravenous administration of a coronary vasodilator, such as Dipyridamole (Persantine™)) or adenosine.

After or during the induction of peak hyperemia, an effective amount of an MR composition comprising Compound ID No. 1, 2, 3 or 4 can be administered to the animal. An MR image of the animal's myocardial tissue after the induction of peak hyperemia can then be acquired. Generally, the acquisition of the image begins at a time frame at least 2 times greater than that required for a first pass distribution of Compound ID No. 1, 2, 3 or 4. In humans, with venous injection of an MR compound, the bolus typically passes through the right heart after approximately 12 sec., and through the left heart after about another 12 sec. Thus, from time of injection to the first pass of the MR compound through the entire heart, approximately 24-30 seconds have passed usually. The second pass of the MR compound usually is seen approximately 45 sec. later. In some embodiments, the MR image of the myocardial tissue of the animal after the induction of peak hyperemia may begin at a time frame at least 5, 10, or 30 times greater than that required for a first pass distribution of the MR compound. Typically, the acquisition of the MR image of the myocardial tissue after the induction of peak hyperemia begins in a time period from about 5 to about 60 minutes after the induction of peak hyperemia. For example, in some embodiments, peak hyperemia is induced in the patient outside of an MR scanner, the MR composition comprising Compound ID No. 1, 2, 3 or 4 is injected at or after peak hyperemia, and the patient is put inside the MR scanner to acquire the MR image of the myocardium after peak hyperemia.

In certain embodiments, the MR images of the myocardium, whether at peak or pre-hyperemia, are T1-weighted images. In some embodiments, T2-weighted images of the myocardium in a pre-hyperemic state are obtained. A T2 weighted image of the myocardium at rest (pre-hyperemic) would give an enhancement of infarcted tissue.

In certain cases, the MR image of the myocardial tissue of the animal in the pre-hyperemic state, if obtained, are compared with the MR image of the myocardial tissue after the induction of peak hyperemia in order to evaluate myocardial perfusion. Zones of abnormal, or low, perfusion will be hypointense (less intense) compared to normal myocardium in the peak hyperemia image.

Certain methods employ a second MR compound. In these methods, peak hyperemia can be induced in an animal and an effective amount of a first MR composition, an MR composition comprising Compound ID No. 1, 2, 3 or 4, is administered. An MR image of the animal's myocardial tissue after the induction of peak hyperemia is acquired, as described previously. An effective amount of a second MR composition can then be administered. In some embodiments, the first and second MR compositions are administered together. The second MR composition may comprise any MR compound including ECF agents or the compounds described herein. Suitable examples of Gd(III)-complexed MR compounds include Gd(III)-DTPA, Gd(III)-DOTA; Gd(III)-DOTAGA; Gd(III)-HP-DO3A, Gd(III)-DTPA-BMA, Gd(III)-DTPA-BMEA, Gd(III)-BOPTA, Gd(III)-EOB-DTPA, Gd(III)-MS-325, Gd(III)-Gadomer-17, or the Gd(III)-complex of the first MR compound administered in the method. Other examples of useful compounds are described in WO 96/23526. The administration of the second MR composition can occur after a time frame sufficient to return the animal to a pre-hyperemic state. For example, the animal may immediately return to a pre-hyperemic state, or the administration of the second compound can occur on a time frame typically ranging from 15 min. to approximately 4 hours after the induction of peak hyperemia. An MR image of the myocardial tissue of the animal in the pre-hyperemic state is then acquired. As one of skill in the art can recognize, the order of the above-referenced steps can be altered, e.g., the administration of the “second” MR composition and acquisition of the pre-hyperemic image can be performed first, while the administration of the “first” MR composition and peak hyperemic scan could be acquired second.

An MR image of the myocardial tissue of the animal in the pre-hyperemic state can be compared with the MR image of the myocardial tissue after the induction of peak hyperemia. Zones of abnormal, or low, perfusion will be hypointense compared to normal myocardium in the peak hyperemia image. Both ischemic and infarct zones appear as hypointense in the peak hyperemia image. In the pre-hyperemic image acquired with the second compound, however, the ischemic zones appear with normal to hyper-intensity, while infarct zones initially appear as hypointense (e.g., after a short time period after injection of the second compound) and then as hyperintense after a longer delay after injection. A comparison of the two images thus allows the characterization of abnormal, or low, perfusion as either ischemia or infarct.

In other methods of evaluating myocardial perfusion, peak hyperemia is induced and an MR composition is administered. An MR image of the animal's myocardial tissue after the induction of peak hyperemia is acquired. The animal is allowed to return to a pre-hyperemic state, and the myocardial tissue is imaged again. The two images can then be compared and examined for zones of ischemia and/or infarct.

Administering an MR composition as described herein (e.g., composition comprising a collagen targeted compound such as one of Compound No. 1, 2, 3, or 4) at peak hyperemia should yield an MR image where healthy tissue is bright, while inducibly ischemic and infarcted tissue is dark, for T1 weighted scans. If there is a dark (hypointense region), one can distinguish whether it is viable tissue (inducible ischemia) or if it is an infarct by comparing the image to an image of the myocardium obtained using one or more of several other methods. For example, one method would be to acquire a T2-weighted scan of the myocardium at rest (e.g., either before or after the induction of peak hyperemia). Infarct appears bright relative to normal compound as described herein (e.g., a collagen targeted MR compound) at rest (pre-hyperemia) and to obtain a pre-hyperemic MR scan of the myocardium, as described previously above; this administration could be performed either before or after the peak hyperemia MR scan. In such a pre-hyperemic scan, normal and inducibly ischemic tissue would enhance, but infarct would not (analogously to nuclear medicine protocols). A third approach would be to administer a composition comprising an extracellular fluid MR compound (ECF), e.g., GdDTPA or GdDOTA, or others as known to those having ordinary skill in the art, at pre-hyperemia, and to obtain an MR image of the myocardium from about 2 to about 60 (e.g., 2 to 20, 2 to 10, 5 to 10, 5 to 20, 10 to 30, 5 to 40, or 8 to 50) minutes after administration of the ECF, e.g., a delayed enhancement image. In this case the infarct would enhance, but the ischemic area would not. Finally, a fourth approach would be to administer a composition comprising an ECF agent at pre-hyperemia and to perform a first pass (MRFP) dynamic perfusion exam to determine if hypointense areas as seen in the targeted MR agent hyperemia scans enhance as quickly and intensely as normal myocardium, which would indicate inducible ischemia.

In one embodiment, method of magnetic resonance (MR) imaging for evaluating myocardial perfusion in an animal comprises (a) inducing peak hyperemia in an animal; (b) administering to the animal an effective amount of an MR composition, the MR composition comprising Compound ID No. 1, 2, 3 or 4; (c) acquiring an MR image of the animal's myocardial tissue after the induction of peak hyperemia in the animal, the acquisition of the MR image beginning at a time frame at least 2 times greater than that required for a first pass distribution of the MR compound; (d) acquiring a second MR image of the animal's myocardial tissue after the induction of peak hyperemia in the animal, the acquisition of the MR image beginning at a time frame at least 4 times greater than that required for a first pass distribution of the MR compound; and (e) evaluating said images of the animal's myocardial tissue to evaluate myocardial perfusion. In some embodiments the method may further comprise acquiring an MR image of the myocardial tissue of the animal in a pre-hyperemic state either before the induction of peak hyperemia in the animal or after a sufficient period of time after the induction of peak hyperemia in the animal to allow the animal to return to a pre-hyperemic state.

In another embodiment, a method of magnetic (MR) imaging for evaluating myocardial perfusion in an animal comprises: (a) inducing peak hyperemia in an animal; (b) administering to the animal an effective amount of an MR composition, the MR composition comprising Compound ID No. 1, 2, 3 or 4; (c) acquiring an MR image of the animal's myocardial tissue after the induction of peak hyperemia in the animal, the acquisition of the MR image beginning at a time frame at least 2 times greater than that required for a first pass distribution of the MR compound; and (d) evaluating said images of the animal's myocardial tissue to evaluate myocardial perfusion. In some embodiments the method may further comprise acquiring an MR image of the myocardial tissue of the animal in a pre-hyperemic state either before the induction of peak hyperemia in the animal or after a sufficient period of time after the induction of peak hyperemia in the animal to allow the animal to return to a pre-hyperemic state.

The compounds of the present disclosure may function to distinguish benign from malignant breast lesions or tumors. Benign lesions such as fibroadenomas and fibrocystic tissue contain significant concentrations of type I collagen. Carcinomas are also collagen rich compared to normal breast tissue which may serve to provide a signature for staging cancer.

In certain embodiments, a compound of the present disclosure (e.g., Compound ID Nos. 1, 2, 3 or 4) may be used. In some embodiments, a T1-weighted imaging is performed after injection of the compound, and a dynamic phase shows all lesions enhanced. The compound is retained in the collagen-rich benign lesions, but washes out of the carcinoma. An image is then acquired at a later time point (e.g., 10 minutes or more post injection) and the benign lesion remains enhanced whereas the carcinoma is not enhanced at this late time point.

In another embodiment, the dynamic contrast-enhanced magnetic resonance imaging (DCE-MRI) approach is used with Compound ID Nos. 1, 2, 3 or 4. Collagen binding alters the signal intensity vs time curve, especially at later time points where the wash-out from the benign lesion is much slower than from the carcinoma.

It is also contemplated that the compounds set forth in this disclosure may be useful in the following applications:

1. Atherosclerosis, High Risk/Vulnerable Plaque.

It has become established that certain atherosclerotic lesions are at risk for rupture, thereby creating a thrombogenic surface. Plaque rupture leads to thrombosis which can result in myocardial infarction or stroke. The precursor lesion of plaque rupture has been defined (Virmani et al, J Interv Cardiol. 2002, 15:439-46) as “thin-cap fibroatheroma” (TCFA). Morphologically, TCFAs have a necrotic core with an overlying thin fibrous cap (<65 mm) consisting of collagen type I, which is infiltrated by macrophages. These lesions are most frequent in the coronary tree of patients dying with acute myocardial infarction. In TCFAs, necrotic core length is approximately 2-17 mm (mean 8 mm) and the underlying cross-sectional luminal narrowing in over 75% of cases is <75% (<50% diameter stenosis). The area of the necrotic core in at least 75% of cases is ≦3 mm². Clinical studies of TCFAs are limited as angiography and intravascular ultrasound (IVUS) catheters cannot precisely identify these lesions. Identification of these precursor lesions of plaque rupture is therefore a great unmet medical need.

Stable lesions, on the other hand, have a thick fibrous (collagenous) cap. The ability to identify and distinguish atherosclerotic plaques based on cap thickness would be of great value. A collagen type I targeted imaging agent such as those described in this application, would bind to the fibrous cap in a collagen-dependent manner. Stable plaques would be seen by T1-weighted MRI as hyperenhanced regions in the lumen and vessel wall. Unstable or at risk plaques (the TCFA) would be seen as a thin hyperenhanced complex zone appearing along the vessel wall.

2. Myocardial Infarct Imaging and Myocardial Viability.

It has been demonstrated that delayed enhancement of infarcted myocardium with GdDTPA enhanced MRI is useful for detecting both transmural and subendocardial infarcts (e.g. Wagner et al. Lancet 2003, 361:374-9). Myocardial infarcts (MI) are typically classified by their EKG response and are grouped into Q-wave MI and non-Q-wave MI. Non-Q-wave infarcts are typically smaller infarcts, however they are associated with a morbidity and mortality associated with larger infarcts. Wagner et al. showed that delayed contrast enhancement MRI was much better at detecting subendocardial infarcts than single photon emission computed tomography (SPECT). Improving the detection of infarct to identify smaller MI would result in a change in treatment for these patients whose MI would otherwise have been missed and would likely improve prognosis. MI results in cardiac remodeling and an increased collagen content. A specific collagen targeted contrast agent would be able to better delineate infarcted regions and improve specificity for infarct.

3. Myocardial Fibrosis—Diagnosis, and Monitoring Response to Therapy.

The extent of myocardial fibrosis is strongly associated with adverse myocardial remodeling, heart failure, life threatening arrhythmias, and early mortality in patients with ischemic and non-ischemic cardiac disorders. A method that allows the identification of early pathological fibrosis and subsequent monitoring of the progression of fibrosis would be useful in identifying at-risk individuals with poor prognosis as well as provide a means for testing the efficacy of new therapies aimed at halting progression of fibrosis. Healthy myocardium is composed of myocardial tissue (80%) with the remaining 20% including the extracellular matrix, is composed of collagen scaffolding.

A hallmark of abnormal cardiac pathology is the expansion of the extracellular volume (ECV) through the development of fibrosis, with increased deposition of type I collagen by cardiac fibroblasts. This occurs in a wide array of cardiac disease including ischemic and non-ischemic cardiomyopathies, which may deposit in different patterns throughout the myocardium. These may either be focal, as found in healed myocardial infarction, or globally distributed throughout the myocardium. As the disease progresses, pathologic fibrosis may be concentrated regionally in addition to being present globally.

Therefore, a specific collagen targeted contrast agent would be ideal for imaging myocardial fibrosis in these patients.

4. Renal Fibrosis—Diagnosis, and Monitoring Response to Therapy.

Renal fibrosis is a final common process of many chronic renal diseases. It is characterized by overdeposition of the extracellular matrix, notable collagen, which eventually leads to the end-stage renal disease (ESRD). Several renal disorders such as diabetic nephropathy, chronic glomerulonephritis, tubulointerstitial fibrosis and hypertensive nephrosclerosis can result into ESRD. Early detection of renal fibrosis would be valuable in order to start treatments earlier and improve the likelihood of reversing the disease. Moreover an imaging agent that allows monitoring of fibrosis would be valuable in assessing response to therapy.

5. Pulmonary Fibrosis—Diagnosis, and Monitoring Response to Therapy.

Pulmonary fibrosis is a pathology whereby the lung tissue becomes scarred with deposits of fibrotic (collagen) tissue. As fibrosis increases there is a decrease in the lung's ability to transfer oxygen to the blood resulting in considerable morbidity and a high likelihood of mortality. There are many causes of pulmonary fibrosis: environmental pollutants/toxins such as cigarette smoke, asbestos; diseases such as scleroderma, sarcoidosis, lupus, rheumatoid arthritis; side effects of radiation treatment or chemotherapy (e.g. bleomycin treatment) for cancer. Early detection and accurate characterization of pulmonary fibrosis can improve patient outcomes. Moreover, as new antifibrotic therapies become available there is a need for means of non-invasively monitoring pulmonary fibrosis and the patient's response to therapy.

6. Liver Fibrosis—Diagnosis, and Monitoring Response to Therapy.

Liver fibrosis is a common result of many diseases which attack the liver: hepatitis B and C; non-alcoholic steatohepatitis (NASH); cirrhosis; primary biliary cirrhosis (PBC); primary sclerosing cholangitis (PSC); and occurs in a fraction of patients with fatty liver. Fibrosis in the liver can be diagnosed but only at an advanced stage with current noninvasive procedures. Biopsy can detect fibrosis at an earlier stage but liver biopsy is not well suited to screening/monitoring disease because of its cost, associated morbidity and known lack of accuracy because of sampling variation, Rockey D C, Bissell D M. “Noninvasive measures of liver fibrosis” Hepatology. 2006 43:S113-20. Early detection and accurate characterization of liver fibrosis can improve patient outcomes. For patients with NASH, diet changes can reverse the disease if caught early enough. Moreover, as new antifibrotic therapies become available there is a need for means of non-invasively monitoring pulmonary fibrosis and the patient's response to therapy.

7. Scleroderma—Diagnosis of Organ Fibrosis and Monitoring Response to Therapy.

Sceloderma is an rare chronic autoimmune disease with an annual incidence of about 20 cases per million in the United States. The disease is characterized by diffuse skin fibrosis, but systemic scleroderma can also affect internal organs. Currently there are no diagnostic tests to enable physicians to determine whether or not fibrosis is spreading to internal organs. Early detection of lung, cardiac or renal fibrosis would enable scleroderma patients to be prioritized for new anti-fibrotic therapies.

### Pharmaceutical Compositions

Pharmaceutical compositions can include any of the compounds described previously, and can be formulated as a pharmaceutical composition in accordance with routine procedures. As used herein, pharmaceutical compositions can include pharmaceutically acceptable salts or derivatives thereof “Pharmaceutically acceptable” means that the agent can be administered to an animal without unacceptable adverse effects. A “pharmaceutically acceptable salt or derivative” means any pharmaceutically acceptable salt, ester, salt of an ester, or other derivative of composition that, upon administration to a recipient, is capable of providing (directly or indirectly) a composition of the present disclosure or an active metabolite or residue thereof. Other derivatives are those that increase the bioavailability when administered to a animal (e.g., by allowing an orally administered compound to be more readily absorbed into the blood) or which enhance delivery of the parent compound to a biological compartment (e.g., the brain or lymphatic system) thereby increasing the exposure relative to the parent species. Pharmaceutically acceptable salts of the compounds or compositions of this disclosure include counter ions derived from pharmaceutically acceptable inorganic and organic acids and bases known in the art, e.g., sodium, calcium, N-methylglutamine, lithium, magnesium, potassium, etc.

Pharmaceutical compositions can be administered by any route, including both oral, intranasal, inhalation, or parenteral administration. Parenteral administration includes, but is not limited to, subcutaneous, intravenous, intraarterial, interstitial, intrathecal, and intracavity administration. When administration is intravenous, pharmaceutical compositions may be given as a bolus, as two or more doses separated in time, or as a constant or non-linear flow infusion. Thus, compositions can be formulated for any route of administration.

Typically, compositions for intravenous administration are solutions in sterile isotonic aqueous buffer. Where necessary, the composition may also include a solubilizing agent, a stabilizing agent, and a local anesthetic such as lidocaine to ease pain at the site of the injection. Generally, the ingredients will be supplied either separately, e.g. in a kit, or mixed together in a unit dosage form, for example, as a dry lyophilized powder or water free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection,” saline, or other suitable intravenous fluids. Where the composition is to be administered by injection, an ampule of sterile water for injection or saline may be provided so that the ingredients may be mixed prior to administration. Pharmaceutical compositions comprise the compounds of the present disclosure and pharmaceutically acceptable salts thereof, with any pharmaceutically acceptable ingredient, excipient, carrier, adjuvant or vehicle.

A pharmaceutical composition is preferably administered to the patient in the form of an injectable composition. The method of administering a compound is preferably parenterally, meaning intravenously, intra-arterially, intrathecally, interstitially or intracavitarilly. Pharmaceutical compositions can be administered to animals including humans in a manner similar to other diagnostic or therapeutic agents. The dosage to be administered, and the mode of administration will depend on a variety of factors including age, weight, sex, condition of the patient and genetic factors, and will ultimately be decided by medical personnel subsequent to experimental determinations of varying dosage followed by imaging as described herein. In general, dosage required for diagnostic sensitivity will range from about 0.1 to 100 mg/kg, preferably between 1 to 40 mg/kg of host body mass. The optimal dose will be determined empirically following the disclosure herein.

### Examples

### Synthesis of Collagen Binding Peptides

Synthetic collagen binding peptides with amidated C-terminii (Table 1, Sequence ID Nos. 1-4) were synthesized using standard solid phase peptide synthesis methods as described herein.

Peptides are synthesized on an automated peptide synthesizer “Liberty Blue” (CEM Inc.) using 1 to 12 batch reactors loaded with 0.1 mmol of commercially available Rink amide resin (˜0.38 mmol/g). A single coupling cycle is used for each amino acid and a 5-fold excess of amino acids is used per coupling to synthesize the peptide on the resin. Standard Fmoc chemistry is used to elongate the peptide on the resin. The Fmoc is removed with a solution of 20% piperidine and 0.1M HOBt in DMF. Each amino acid is dissolved in DMF to give a 0.2 M solution and is coupled to the peptide using a 0.5 M solution of diisopropylcarbodiimide in DMF, and 1.0M Oxyma (or HOBt) After each deprotection or coupling step the resin is washed three times with DMF. The completed peptide/resin is washed with 1:1 DCM:CH₂Cl₂ and transferred back to the falcon tube, in ca. 20 mL of 1:1 DCM:CH₂Cl₂ mixture.

After the synthesis of the peptide on the resin is complete, the peptide is filtered and subsequently cleaved from the resin using the following cleavage cocktail: TFA/TIS/H₂O 95:2.5:2.5 (10 mL per 100 μmoles of peptide). The solution of fully deprotected peptide is precipitated with diethyl ether (40 mL). The peptide solid is isolated after centrifugation and decantation and then re-dissolved in a 1:1 mixture of DMSO/10 mM NH₄OAc (ca. 40 mL). The cyclization is monitored by LC-MS (24 h). The cyclic peptide is purified by reverse phase preparative HPLC on a C-5 column using a gradient of 5% mobile phase A (0.1% TFA in water) to 60% mobile phase B (0.1% TFA in acetonitrile) over 23 minutes. The fractions of pure peptide are pooled and lyophilized to give the final peptide moiety.

### Synthesis of Collagen Binding Compounds

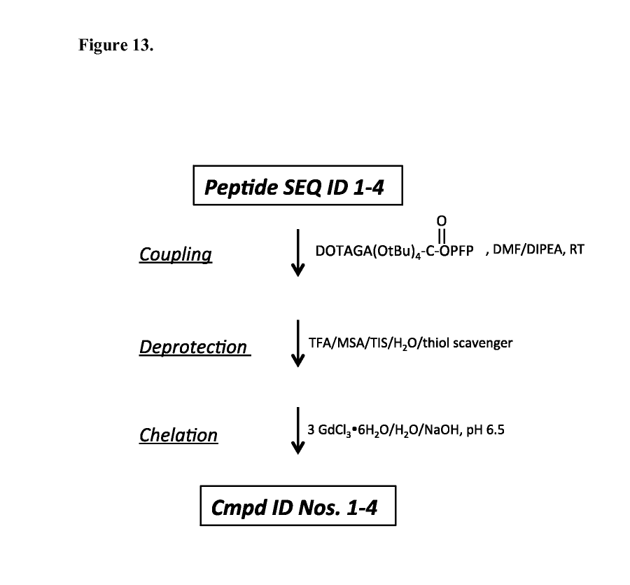

Procedure for Preparing the Gd-DOTAGA Peptide Conjugate Compounds (FIG. 13, Compound ID Nos. 1-4)

Chelate Coupling:

The cyclized peptide (0.05 mmol) containing N primary amines is dissolved in DMF (15 ml). t-butyl protected DOTAGA-pentafluorophenylester (1.2×N primary amines×0.05 mmol) is added and the pH (measured on wet pH stick) of the reaction mixture adjusted to 6.5-7.5 with di-isopropylethylamine (DIEA). The reaction is stirred overnight at room temperature and upon completion of the reaction, verified by LC/MS is then triturated with brine and washed with water to give a solid. The solid is filtered and dried under vacuum overnight. Purity and identity are confirmed by LC-MS and the product is used without further purification.

Deprotection:

The crude product, protected DOTAGA-peptide conjugate, is dissolved in a mixture of TFA/methanesulfonic acid/TIS/water/dodecanethiol (or 2,2-ethylenedioxide-diethanethiol) (20 ml, 18:0.5:0.5:0.5:0.5) and stirred for ca. 4 hr at 40 C and then poured into ether giving a white precipitate. The precipitate is isolated by filtration and washed with ether (2×20 mL) The crude DOTAGA-peptide ligand conjugate is purified by reverse phase preparative HPLC on a phenomenex C-5 Luna column using a gradient of 5% mobile phase A (0.1% TFA in water) to 25% mobile phase B (0.1% TFA in acetonitrile) over 20 minutes, and held at 25% B for 10 minutes. The fractions of pure peptide are pooled and lyophilized to give the final peptide chelate conjugate. Purity and identity are confirmed by LC/MS.

Chelation:

The purified peptide ligand conjugate is dissolved in H₂O (20 ml/g peptide conjugate) and the pH adjusted to 6-7 with a 1 N NaOH solution. Solid GdCl₃.6H₂O (1.1×N primary amines×0.05 mmol peptide) is dissolved in water (ca 1 ml/100 mg) and added at RT. The pH is re-adjusted to 6-7 with 1.0 N NaOH. The reaction can be complete in 4 hrs, but can also be stirred overnight. The chelation reaction is checked by LC-MS to ensure that it has gone to completion, usually resulting in a cloudy suspension. A solution of 100 mM EDTA (to scavenge the excess gadolinium ions) is added dropwise with stirring until the solution becomes clear, pH must be maintained at 6-7 during EDTA addition.

Purification:

The crude product is purified by preparative HPLC (Phenomenex C-5 Luna, water/ACN 10% water to 30% Acetonitrile over 30 minutes. Fractions are pooled, lyophilized and the purified product analyzed by LC-MS.

C. Synthesis of Compound ID No. 1.

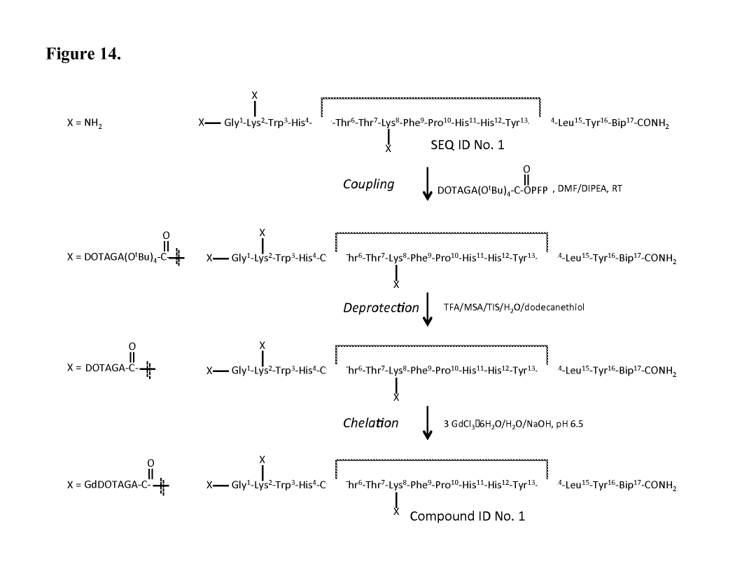

See reaction scheme shown in FIG. 14. Peptide, SEQ ID No. 1 (1.05 mmol) was dissolved in 50 mL of DMF. t-butyl protected DOTAGA-pentafluorophenyl ester (3.8 mmol) was added and DIEA added to adjust the pH to 7.0. After reaction at room temperature overnight, the reaction was triturated with brine (200 mL) and washed with water, and partially dried on filter. The crude wet solid was dissolved in a 60 mL mixture of TFA:methane sulfonic acid:TIS:water:2,2-diethylinedioxy-diethane thiol (55:1:1:2:1) and stirred for 2-3 hours at 40 C. The deprotected ligand, Compound ID No. 5, was obtained after precipitation with diethyl ether. The crude solid was then taken up in 60 mL water and purified by preparatory HPLC, using phenomenex C-5 Luna column using a gradient of 5% mobile phase A (0.1% TFA in water) to 25% mobile phase B (0.1% TFA in acetonitrile) over 20 minutes, and held at 25% B for 10 minutes. The fractions of pure compound are pooled and lyophilized to give the final peptide moiety, purity and identity confirmed by LC/MS (48% yield).

The solid is dissolved in ca. 40 mL water and neutralized by addition of 1 M NaOH until the pH was 6.5. Solid GdCl₃.6H₂O (600 mg, 1.6 mmol) was dissolved in ca. 5 ml water and added at RT. The pH is re-adjusted to 6.5 with 1.0N NaOH. The solution was stirred overnight and the resultant solution was cloudy. Na₂H₂EDTA solution (0.1 M) was added dropwise with stirring until the solution became clear. The pH was maintained at 6-7 with 1.0 N NaOH The resultant clear solution was purified by preparative HPLC (Phenomenex C5 Luna, water and ACN gradient and the product, at 28-32% ACN. The product Compound ID No. 1 was lyophilized leaving 1.6 g of white powder which was analyzed by LC-MS and gave the correct mass [(M+3)/3] of 1360.4

TABLE 3
Summary of analytical data and yields of Compound ID No. 1
batches

| | Isolated | | HPLC | | Mass |
|---|---|---|---|---|---|
| | Yield (grams | | Retention | Purity | Spec |
| Batch # | chelate) | % Yield¹ | Time (min) | (%) | (M + 4)/4 |
| 1 | 0.44 | 32 | 12.85 | 98 | 1360.1 |
| 2 | 1.1 | 40 | 12.8 | 97 | 1360.6 |
| 3 | 1.6 | 53 | 12.88 | 98 | 1360.65 |
| 4 | 2.44 | 60 | 12.84 | 98 | 1360.4 |
| 5 | 1.2 | 30 | 12.85 | 97 | 1360.5 |
| 6 | 2.1 | 52 | 12.8 | 97 | 1360.4 |
| 7 | 1.95 | 48 | 12.86 | 96 | 1360.8 |
| 8 | 0.8 | 13 | 12.87 | 96 | 1360.3 |

¹Based on cyclic peptide starting material

The following additional compounds were synthesized by derivatizing the collagen binding peptide with GdDOTAGA using the following general procedure:

2. Compound ID No. 2 was prepared using peptide SEQ ID No. 2 following the general procedure above to give 12.5 mg of product with the correct molecular mass. The C-terminus is capped with an —NH₂ amide and GdDOTAGA is linked to the peptide terminal nitrogen and lysine epsilon amino groups through an amide bond.

3. Compound ID No. 3 was prepared using peptide SEQ ID No. 3 following the general procedure above to give 9.5 mg of product with the correct molecular mass. The C-terminus is capped with an —NH₂ amide and GdDOTAGA was linked to the peptide terminal nitrogen and lysine epsilon amino groups through an amide bond.

4. Compound ID No. 4 was prepared using peptide SEQ ID No. 4 following the general procedure above to give 5.8 mg of product with the correct molecular mass. The C-terminus is capped with an —NH₂ amide and GdDOTAGA was linked to the peptide terminal nitrogen or lysine epsilon amino groups through an amide bond.

### Relaxivity of Compound ID Nos. 1, 2, and 4

The relaxivity of Compound ID Nos. 1, 2 and 4 were determined in PBS at 37° C. using a Bruker mq60 spectrometer operating at 60 MHz (1.4 tesla). Samples were equilibrated at concentrations ranging from 0-200 μM for at least 30 minutes at 37° C. T₁ was measured using an inversion recovery sequence. Relaxivities were calculated by subtracting the relaxation rate of the buffer with Gd from the relaxation rate of the buffer sample with Gd and then dividing the result by the concentration of Compound. The relaxivities determined this way are shown in Table 4.

TABLE 4
Relaxivities of selected Compound ID Nos.

| CMPD | SEQ | | | C- |
|---|---|---|---|---|
| ID | ID | R1^{a} | Peptide Sequence^{b} | term |
| 1 | 1 | 43.4 | G.K.W.H.C.T.T.K.F. | NH₂ |
| | | | P.H.H.Y.C.L.Y.Bip | |
| 2 | 2 | 42.9 | K.W.H.C.T.T.K.F.P. | NH₂ |
| | | | H.H.Y.C.L.Y.Bip | |
| 3 | 3 | N/A | K.Y.W.H.C.T.T.K.F. | NH₂ |
| | | | P.H.H.Y.C.L.Y.Bip | |
| 4 | 4 | 51.2 | K.W.H.C.Y.T.K.F.P. | NH₂ |
| | | | H.H.Y.C.V.Y.Bip | |

^{a}mM-1s-1, pH 7.4 PBS at 1.4 T, 37° C.
^{b}Bip = L-4,4′-biphenylalanine

### Collagen Binding Properties of Compounds of the Invention

Preparation of Human Collagen:

10 ml of a solution of 3 mg/ml of human type I collagen (VitroCol solution, Advanced Biomatrix, cat#5007-A) is dialyzed against 10 mM Phosphate (NaH₂PO₄), pH 4.2 at 4° C. with three changes of the dialysis buffer. The protein concentration is determined by liquid chromatography determination of hydroxyproline (P. Hutson, J. Chromatogr. B, 791 (2003) 427-430).

Preparation of Rat Collagen:

10 ml of a 3.79 mg/mL solution of rat collagen (acid soluble, type I, rat tail, Millipore Inc, cat#08-115) is dialyzed against 10 mM Phosphate (NaH₂PO₄), pH 4.2 at 4° C. with three changes of the dialysis buffer.

Preparation of Canine Collagen:

Canine collagen (Native canine Collagen Type I and III protein, YO protein AB, cat#739) is dissolved in 0.5 M acetic acid at 3.3 mg/ml by vortexing and shaking overnight at 4° C. The solution is then dialyzed against 10 mM Phosphate (NaH₂PO₄), pH 4.2 at 4° C. with three changes of the dialysis buffer.

Preparation of Microtiter Plate:

Ice-cold 1×PBS pH 10.8 is added to the collagen solution for a final collagen concentration of 10 μM, pH 7.4. Collagen solutions are gelled and dried down in the wells of a 96 well microtiter plate (Corning Polystyrene Flat Bottom, cat#3641). 70 μl of 10 μM collagen is aliquoted into each well in every other lane in the plate (48 wells) and the plate is incubated at 37° C. for 18 hours to form a gel and evaporate to dryness. Ungelled collagen is removed by washing the collagen films with 200 μl 1×PBS pH 7.4 (four times, 15 min per wash). The thin collagen fibril film remains, coating the bottom of each well. After washing by PBS the plate is again dried at 37° C. for 2 hours and is stored at −20° C. The final well content of gelled collagen is measured by determination of hydroxyproline and is around 180 μg/ml.

Collagen Binding Assay:

a serial dilution of 0.2 μM-30 μM of the peptide chelate is prepared in PBS, pH 7.4 (˜300 μL of solution for each concentration). 90 μl of each concentration is also reserved in a HPLC glass vial as a sample to measure the total concentration. 140 μL of each dilution of peptide chelate is added to wells containing and non-containing collagen (control for nonspecific plastic binging). The plate is then incubated on a shaker table (300 rpm) for 2 hours at room temperature to allow the compound to bind. After 2 hours the supernatant from each well (with or without collagen) is transferred to an HPLC glass vial. The concentration of free, unbound compound in the sample supernatants and the concentration of compound in the reserved (total) sample are determined by ICP-MS (Agilent 7500, gadolinium concentration). The concentration of compound bound to collagen is determined as [bound]=[total]−[unbound].

Collagen Binding Constant:

The binding of compounds to human, rat and dog collagen (5 μM) was measured over the concentration range 0.2-5 μM of Comp ID Nos.: 1-4. The binding data was fit to a model of 1 binding site. This yielded dissociation constants (Kd) as indicated in Table 5.

TABLE 5
Collagen binding of compounds to human,
rat and dog collagen, 37° C., pH 7.4

| | | Collagen | | | | |
|---|---|---|---|---|---|---|
| | | Binding (uM) | | | | |
| CMPD | SEQ | Human | Rat | Dog | | C- |
| ID | ID | (K_{d}) | (K_{d}) | (K_{d}) | Peptide Sequence¹ | term |
| 1 | 1 | 3.8 | 21.1 | 4.4 | G.K.W.H.C.T.T.K.F. | NH₂ |
| | | | | | P.H.H.Y.C.L.Y.Bip | |
| 2 | 2 | 4.9 | 20.6 | 4.9 | K.W.H.C.T.T.K.F.P. | NH₂ |
| | | | | | H.H.Y.C.L.Y.Bip | |
| 3 | 3 | 29.8 | N/A | N/A | K.Y.W.H.C.T.T.K.F. | NH₂ |
| | | | | | P.H.H.Y.C.L.Y.Bip | |
| 4 | 4 | 9.6 | 4.6 | 2 | K.W.H.C.Y.T.K.F.P. | NH₂ |
| | | | | | H.H.Y.C.V.Y.Bip | |

¹Bip = L-4,4′-biphenylalanine

### Pharmacokinetics (Rat)

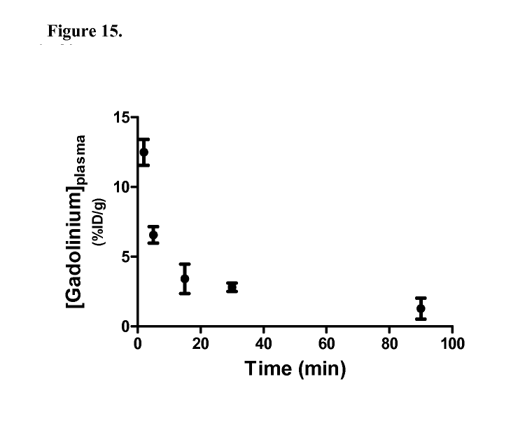

Compound ID No. 1 was formulated at pH 7 in 80 mM sucrose and administered to Sprague Dawley rats (n=2) at dose of 1.3 umol/kg using a bolus IV injection. Plasma was sampled at 2, 5, 15, 30, and 90 minutes post-injection and analyzed for gadolinium content using ICP-MS (FIG. 15). The pharmacokinetic profile was characterized by a rapid initial blood clearance.

Uptake into Fibrotic Myocardial Tissue

The uptake of Compound ID Nos. 1, 2 and 4 into myocardial fibrotic tissue was determined in a rat model of healed myocardial infarction by comparing uptake in normal vs. scarred myocardium. The collagen binding peptide chelate conjugates have greater binding in fibrotic cardiac tissue as compared with normal myocardial tissue

Myocardial infarction was induced in Sprague Dawley rats by occlusion of the left anterior descending coronary artery followed by reperfusion. The rats were anesthetized with an intraperitoneal (i.p.) injection of 100 μg pentobarbital sodium per gram body weight and a thorocotamy was performed. The pericardium was removed and the left anterior artery was sutured with a 7.0 silk suture for 60 minutes after which reperfusion was established.

Compound ID Nos. 1, 2, and 4 were injected into separate animals 3 weeks following infarction at a dose of ˜1 umol/kg. Animals were sacrificed at 60 minutes post-injection and the heart removed and sectioned for analysis. Tissue samples from normal myocardium and infarcted myocardium were analyzed for gadolinium and hydroxyproline (collagen) content (Table 6).

TABLE 6
Concentration of gadolinium and collagen (as measured by
hydroxyproline, Hyp, content) in healthy and infarcted heart tissue
following administration of Compound ID Nos. 1, 2 and 4.

| CMPD ID | | | [Gd]ₜᵢₛₛᵤₑ | [Hyp]ₜᵢₛₛᵤₑ |
|---|---|---|---|---|
| No. | Rat | Tissue | (nmol/g) | (μg/g) |
| 1 | G2R6 | healthy | 0.368 | 663 |
| | | heart | 0.390 | 537 |
| | | | 0.411 | 491 |
| | | infarcted | 0.926 | 5439 |
| | | heart | 0.856 | 4424 |
| | G2R7 | healthy | 0.357 | 950 |
| | | heart | 0.316 | 1190 |
| | | | 0.391 | 1762 |
| | | infarcted | 0.793 | 4797 |
| | | heart | 0.900 | 6715 |
| | G2R8 | healthy | 0.294 | 573 |
| | | heart | 0.240 | 539 |
| | | | 0.291 | 490 |
| | | infarcted | 0.719 | 3874 |
| | | heart | 0.655 | 2907 |
| | | | 0.265 | 916 |
| | | | 0.234 | 565 |
| | | infarcted | 0.598 | 4866 |
| | | heart | 0.636 | 5926 |
| | G2R10 | healthy | 0.206 | 712 |
| | | heart | 0.227 | 590 |
| | | | 0.199 | 638 |
| | | infarcted | 0.537 | 6979 |
| | | heart | 0.568 | 6412 |
| 2 | G3R11 | healthy | 0.349 | 552 |
| | | heart | 0.314 | 464 |
| | | | 0.438 | 920 |
| | | infarcted | 1.012 | 3688 |
| | | heart | 0.984 | 3378 |
| | G3R12 | healthy | 0.442 | 592 |
| | | heart | 0.489 | 880 |
| | | | 0.457 | 784 |
| | | infarcted | 1.293 | 4016 |
| | | heart | 1.198 | 3339 |
| | G3R13 | healthy | 0.451 | 573 |
| | | heart | 0.596 | 1449 |
| | | | 0.533 | 1042 |
| | | infarcted | 2.028 | 8828 |
| | | heart | 1.639 | 5735 |
| | G3R14 | healthy | 0.497 | 571 |
| | | heart | 0.573 | 639 |
| | | | 0.497 | 602 |
| | | infarcted | 2.442 | 7247 |
| | | heart | 2.229 | 5400 |
| | G3R15 | healthy | 0.151 | 833 |
| | | heart | 0.179 | 963 |
| | | | 0.164 | 608 |
| | | infarcted | 0.485 | 5390 |
| | | heart | 0.526 | 5601 |
| 4 | G4R16 | healthy | 0.373 | 496 |
| | | heart | 0.547 | 904 |
| | | | 0.446 | 660 |
| | | infarcted | 1.327 | 3569 |
| | | heart | 1.435 | 4247 |
| | G4R17 | healthy | 0.513 | 614 |
| | | heart | 0.517 | 615 |
| | | | 0.572 | 716 |
| | | infarcted | 2.786 | 7767 |
| | | heart | 2.844 | 7374 |
| | G4R18 | healthy | 0.418 | 657 |
| | | heart | 0.411 | 585 |
| | | | 0.375 | 525 |
| | | infarcted | 1.706 | 4431 |
| | | heart | 1.477 | 3496 |
| | G4R19 | healthy | 0.406 | 605 |
| | | heart | 0.446 | 684 |
| | | | 0.486 | 762 |
| | | infarcted | 1.653 | 4860 |
| | | heart | 2.233 | 6656 |
| | G4R20 | healthy | 0.391 | 594 |
| | | heart | 0.467 | 833 |
| | | | 0.543 | 954 |
| | | infarcted | 1.728 | 4393 |
| | | heart | 2.129 | 5957 |

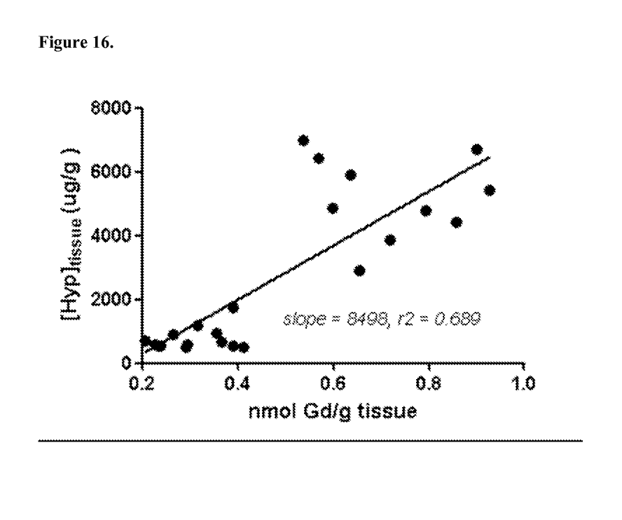

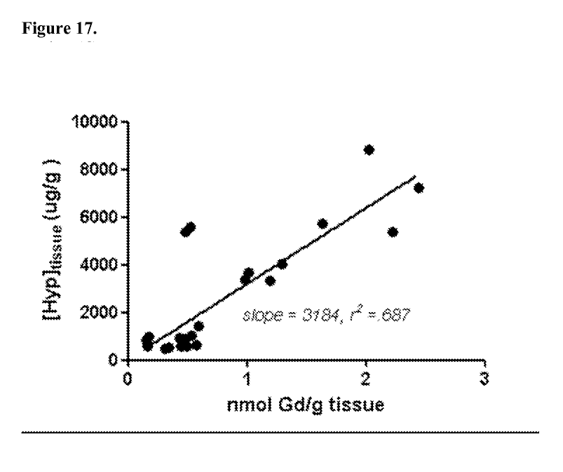

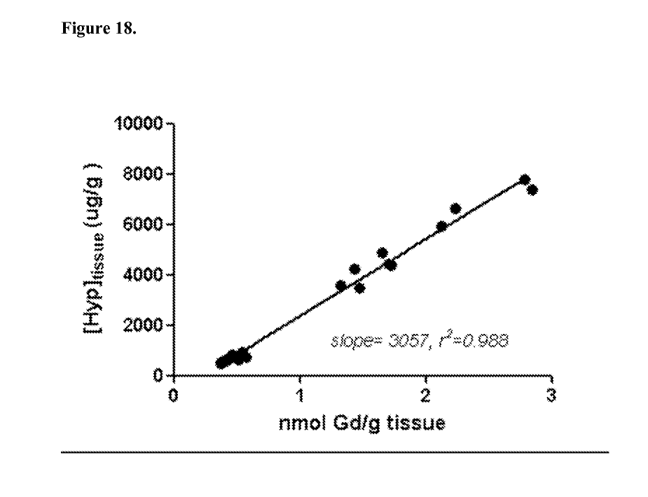

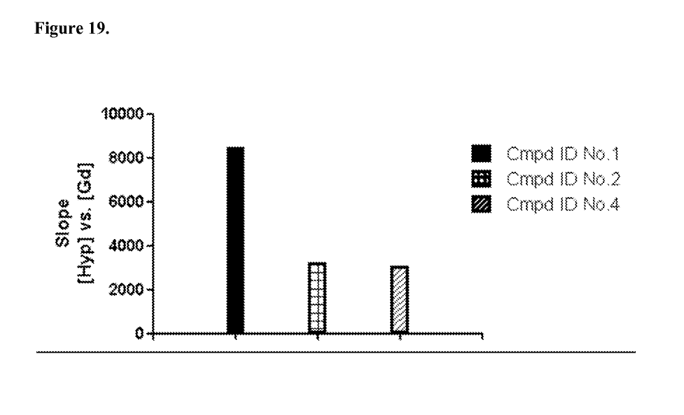

There is a linear relationship between gadolinium concentration and collagen tissue content (measured by assessing hydroxyproline concentration) for all compounds tested. As the concentration of collagen in tissue increases the concentration of collagen binding peptide chelate conjugate compound should also increase. The slope for this correlation is a measure of efficacy. Compounds exhibiting a greater slope for collagen vs. concentration of collagen binding peptide chelate conjugate compound will exhibit a greater dynamic range for imaging fibrosis and the higher slope will translate into the ability to more accurately stage fibrosis. The calculated slope for Compound ID No. 1 was 8498 (FIG. 16) was superior to the other compounds tested including Compound ID No. 2 (slope=3184, FIG. 17), and Compound ID No. 4 (slope=3057, FIG. 18) in the parallel experiment indicating that Compound ID No. 1 binds to the myocardial collagen with significantly greater sensitivity compared to all of the other compounds (FIG. 19).

### Myocardial Perfusion Imaging

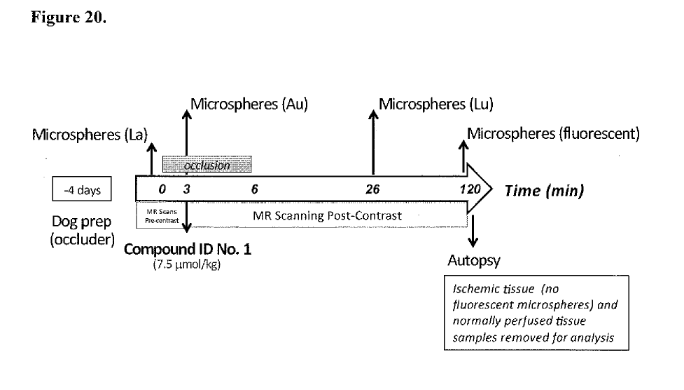

To mimic severe ischemia, a canine model was used in which an inflatable variable vascular occluder was placed around the left anterior descending coronary artery (LAD) to allow occlusion and reperfusion. Imaging was performed on a 3 T clinical scanner 4 days after implantation of the occluder. The conventional saturation recovery pulse sequence for stress perfusion imaging was compared with a segmented inversion method. The purpose of the segmented inversion method was to leverage the steady-state properties of Compound ID No. 1. This segmented inversion recovery pulse sequence provides greater T1 weighting, higher spatial resolution, and greater myocardial tissue contrast. Additionally, since imaging is delayed, the entire heart can be imaged. After baseline MRI scanning, the balloon was inflated. Compound ID No. 1 was administered as an i.v. bolus at a dose of 7.5 μmol/kg one minute after coronary artery occlusion. The occlusion was maintained for an additional 4 minutes, after which blood flow was restored. Imaging was performed prior to occlusion release and at multiple time points following reperfusion (up to 120 minutes after reperfusion, see FIG. 20)

To assess relative perfusion, labeled microspheres were administered at 3 timepoints in the study. La-labeled microspheres were given before coronary artery occlusion, Au-labeled microspheres were given during coronary artery occlusion, and Lu-labeled microspheres were given after reperfusion. In addition, prior to euthanasia, the variable occluder was re-inflated and fluorescent microspheres were administered in combination with KCl to arrest the heart and visually delineate the area of hypo-perfusion. The animal was sacrificed at ca. 120 minutes post Compound ID No. 1 and the heart removed and sectioned according to American Heart Association guidelines (M D Cerqueira et al, Circulation, 2002, 105:539-42). At autopsy under ultraviolet light, the hypo-perfused myocardium was differentiated from normal myocardium due to the lack of fluorescence in the hypo-perfused tissue. Based on the fluorescence, a sample of the ischemic territory and a sample of the remote myocardium were taken for ex vivo analysis. These tissue samples were weighed, digested in nitric acid, and analyzed for the elements La, Au, Lu (the three microsphere injections) and gadolinium (Compound ID No. 1) using ICP-MS. Concentrations in the ischemic tissue were compared to that of the remote myocardium to assess regional flow and probe uptake.

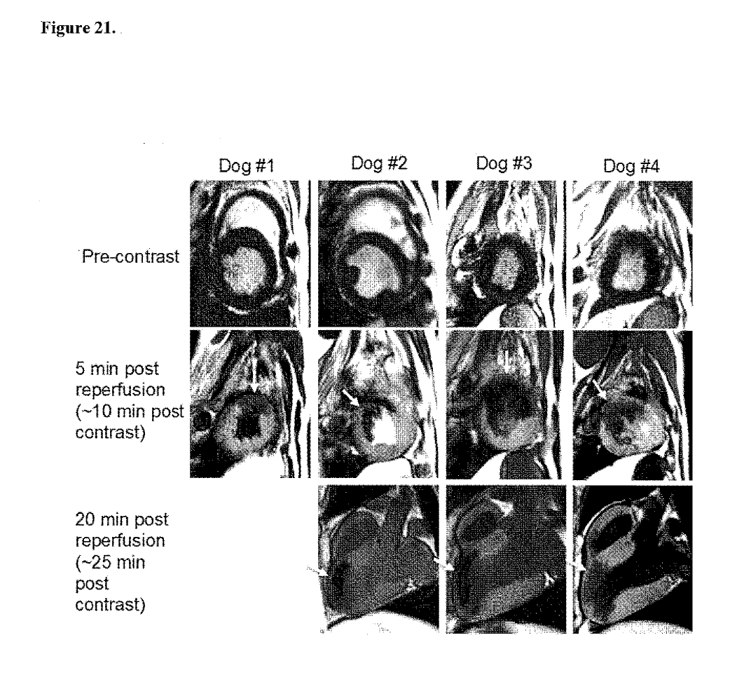

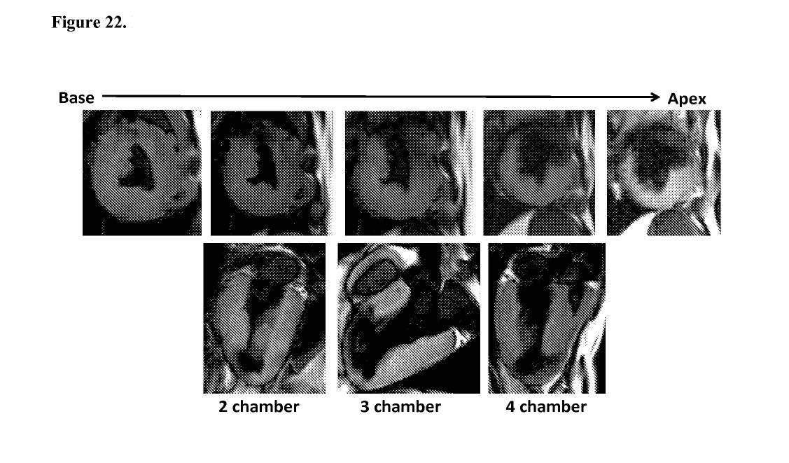

The myocardial perfusion defect was readily visualized following administration of Compound ID No. 1. The optimal sequence for visualizing the hypoperfused area was the inversion recovery sequence, which was able to visualize the perfusion defect longer and with higher conspicuity than the saturation recovery sequence (FIGS. 21, 22).

Prior to Compound ID No. 1 injection, the myocardium and ventricles are both dark. Ten minutes after injection the ventricles are hyperintense because of contrast agent in the blood and the myocardial perfusion defect (ischemic area) is visualized as a dark zone (orange arrow) while the normal myocardium is seen with bright signal. At 20 minutes, the signal in the blood has decreased but the myocardium remains dark in the ischemic zone and brighter in normal myocardium.

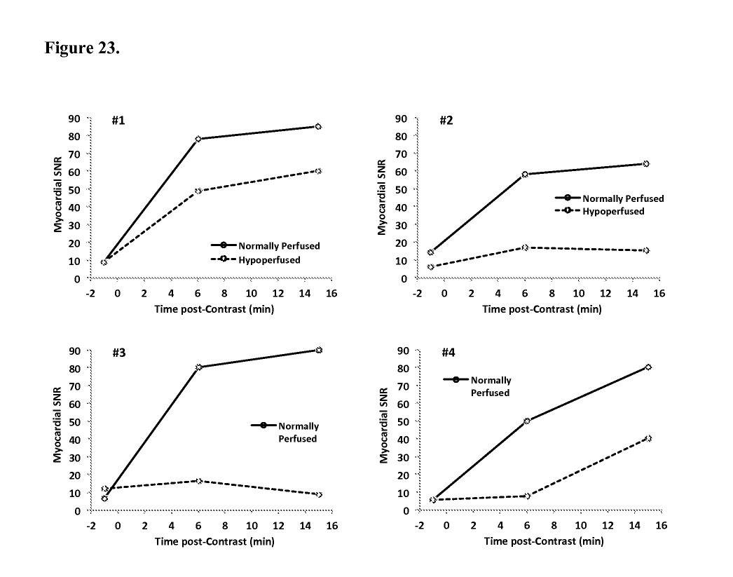

Data were quantitatively evaluated at pre-contrast, at 6 minutes post-contrast, and 15 minutes post-contrast using signal-to-noise ratios (SNR) for normally perfused myocardium, SNR for hypo-perfused myocardium, and contrast to noise ratio (CNR) for normal-to-hypoperfused myocardium, Tables 8-10 and FIG. 23. In the normally perfused myocardium there is a strong and significant increase in SNR following Compound ID No. 1 injection and this enhancement persists at the 15 min time point (P<0.01 compared to pre-contrast; note all comparisons were made using repeated measures ANOVA with a Bonferroni correction for multiple, pairwise comparisons). On the other hand the increase in SNR post Compound ID No. 1 in the hypoperfused myocardium was not significantly higher compared to the pre-contrast image. This resulted in an extremely high contrast between normal and hypoperfused myocardium following Compound ID No. 1 injection with CNR=49±16 at 6 minutes versus 2.7±3.1 pre-injection (P=0.005) and CNR=58±21 at 15 minutes (P=0.002 versus pre-injection).

TABLE 7
Summary of image data analysis (SNR in normal
and hypoperfused myocardium, CNR between
normal and hyperperfused tissue) in animals with
temporary coronary artery occlusion (Compound ID No. 1)

| Characteristic | N | Mean | S.D. | Minimum | Maximum |
|---|---|---|---|---|---|
| Normally Perfused | | | | | |
| Myocardial SNR | | | | | |
| Pre-contrast | 4 | 6.3 | 6.7 | 0.8 | 15.9 |
| 6 min post-contrast | 4 | 34.1 | 17.7 | 10.0 | 49.7 |
| (COMPOUND ID No. 1) | | | | | |
| 15 min post-contrast | 4 | 36.1 | 10.9 | 20.0 | 42.8 |
| Hypoperfused Myocardial | | | | | |
| SNR | | | | | |
| Pre-contrast | 4 | 4.6 | 2.7 | 0.8 | 6.8 |
| 6 min post-contrast | 4 | 13.1 | 12.9 | 1.6 | 31.2 |
| 15 min post-contrast | 4 | 13.6 | 11.3 | 3.9 | 30.0 |
| Normally/Hypoperfused | | | | | |
| Myocardial CNR | | | | | |
| Pre-contrast | 4 | 2.7 | 3.1 | −0.3 | 6.6 |
| 6 min post-contrast | 4 | 48.8 | 15.7 | 34.6 | 71.0 |
| 15 min post-contrast | 4 | 57.5 | 21.0 | 35.0 | 85.0 |

Comparisons over time were made using repeated measures ANOVA with a Bonferroni correction for multiple, pairwise comparisons.

TABLE 8
Normally Perfused Myocardial SNR

| Comparison | Mean ± SD | P Value |
|---|---|---|
| Pre-contrast versus 6 min | 6.3 ± 6.7 vs 34.1 ± 17.4 | 0.006 |
| post-contrast | | |
| Pre-contrast versus 15 min | 6.3 ± 6.7 vs 36.1 ± 10.9 | 0.004 |
| post-contrast | | |
| 6 min post-contrast versus | 34.1 ± 17.4 vs 36.1 ± 10.9 | 1.00 |
| 15 min post-contrast | | |

TABLE 9
Hypoperfused Myocardial SNR

| Comparison | Mean ± SD | P Value |
|---|---|---|
| Pre-contrast versus 6 min | 4.6 ± 2.7 vs 13.1 ± 12.9 | 0.37 |
| post-contrast | | |
| Pre-contrast versus 15 min | 4.6 ± 2.7 vs 13.6 ± 11.3 | 0.33 |
| post-contrast | | |
| 6 min post-contrast versus | 13.1 ± 12.9 vs 13.6 ± 11.3 | 1.00 |
| 15 min post-contrast | | |

TABLE 10
Normal/Hypoperfused Myocardial CNR

| Comparison | Mean ± SD | P Value |
|---|---|---|
| Pre-contrast versus 6 min | 2.7 ± 3.1 vs 48.8 ± 15.7 | 0.005 |
| post-contrast | | |
| Pre-contrast versus 15 min | 2.7 ± 3.1 vs 57.5 ± 21.0 | 0.002 |
| post-contrast | | |
| 6 min post-contrast versus | 48.8 ± 15.7 vs 57.5 ± 21.0 | 1.00 |
| 15 min post-contrast | | |

Reduction in flow was verified by microspheres administered through the left atrial catheter during occlusion. (Spuentrup, et al., Circulation, 2009, 1768-75). During coronary occlusion, flow to the ischemic territory was only 22% (P<0.00001) of that to the remote, non-ischemic myocardium. Tissue samples were also analyzed for gadolinium (Gd) as a marker of Compound ID No. 1 content. The concentration of Gd in the ischemic tissue was 79% of the value in the normal myocardium (P=0.04). This difference was quite remarkable given that the animals were sacrificed ˜2 hours after Compound ID No. 1 injection. That is, even with ˜2 hours available for redistribution, Compound ID No. 1 still showed preferential deposition in normal vs ischemic cardiac tissue.

In 2009, Spuentrup et al. described as similar study in a pig model using a collagen binding compound called EP-3600 (Spuentrup, et al., Circulation, 2009, 1768-75). Spuentrup et al. used a dose of 12.3 μmol/kg which is higher than the dose of Compound ID No. 1 used herein (7.5 μmol/kg). Spuentrup et al. observed a significant increase in CNR between normal and hypoperfused myocardium that was on the order of ΔCNR=15 measured at 5 or 20 minutes post injection. In the current study with Compound ID No. 1 the ΔCNR was found to be 3 to 4-fold higher, even though the dose of Compound ID No. 1 is 40% lower than the dose of compound EP-3600 used in the Spuentrup paper.

These data demonstrate that Compound ID No. 1 provide MR images reflective of perfusion in the myocardium. The collagen targeted contrast agent provides positive image contrast in the normally perfused myocardium, whereas the ischemic part of the myocardium is hypointense (dark).

### Myocardial Fibrosis Imaging

An experimental protocol was developed to test the ability of Compound ID No. 1 to differentiate acute from chronic myocardial infarction in an in-vivo large animal (canine) model. In this model, early following acute myocardial infarction, pathologic fibrosis has not fully developed within the infarct zone (fibrosis-poor), whereas in chronic MI (˜8 weeks following MI), dense fibrosis fully replaced necrotic myocardium (fibrosis-rich).

In the myocardial fibrosis canine model, a vascular occluder was placed surgically around the left anterior descending coronary artery (LAD) to allow occlusion and reperfusion. Two animals were studied following acute MI and two additional animals were studied both following acute MI, and chronically at 8 weeks. The LAD vessel was completely occluded for 70-90 minutes and then released to allow reperfusion. The chest was sutured closed, and the animal was allowed to recover. Imaging was performed on a 3 T clinical scanner in an acute (<1 week, minimal fibrosis expected in acute necrosis) and chronic (8 weeks, healing complete, necrotic myocardium replaced by dense collagenous scar) time point after infarction. For each time point, the animals undergo 2 scans separated by 48 hours with conventional gadolinium contrast (0.2 mmol/kg GdDTPA) and Compound ID No. 1 (0.0075 mmol/kg).

Imaging Parameters

Imaging was performed on a 3 T clinical scanner (Siemens Verio). Breath holding was achieved by temporarily turning off the animal ventilator, and all images were ECG gated. Standard short and long axis cine imaging was performed throughout the left ventricle to identify left ventricular function and regional wall motion abnormalities. Delayed enhancement imaging (segmented 2D inversion recovery gradient echo) was performed prior to and serially following Compound ID No. 1 administration. Two strategies were employed: (1) a fixed inversion time was chosen to null pre-contrast myocardium (TI˜650 ms), and (2) a variable inversion time set to null post-contrast myocardium as is performed in traditional delayed enhancement imaging.

After imaging, animals were euthanized and post mortem analysis of myocardium was performed. Tissue was assessed grossly by histopathologic staining with triphenyltetrazolium chloride for myocardial infarction, and microscopically with Masson's trichrome staining for fibrosis. Additionally quantitative tissue analysis for hydroxyproline was used to measure total collagen content in tissue and compared to tissue gadolinium concentration by inductively coupled plasma-mass spectrometry. MR data was analyzed quantitatively for conspicuity of infarction and image quality as well as contrast to noise ratio (CNR) for infarct-normal myocardium and infarct-blood.

Image Analysis Results

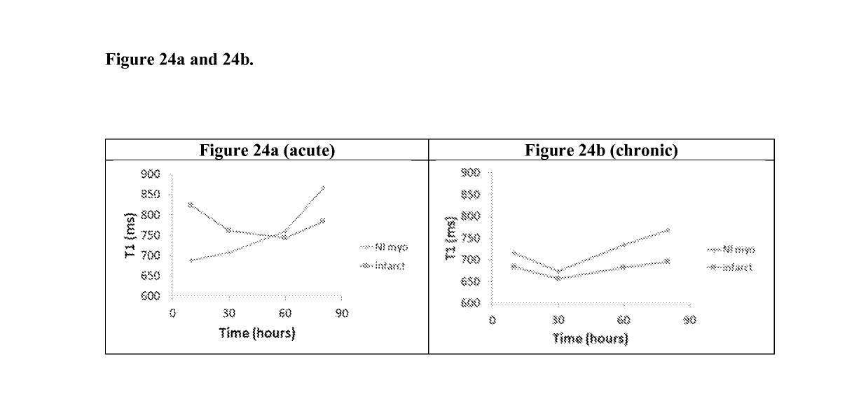

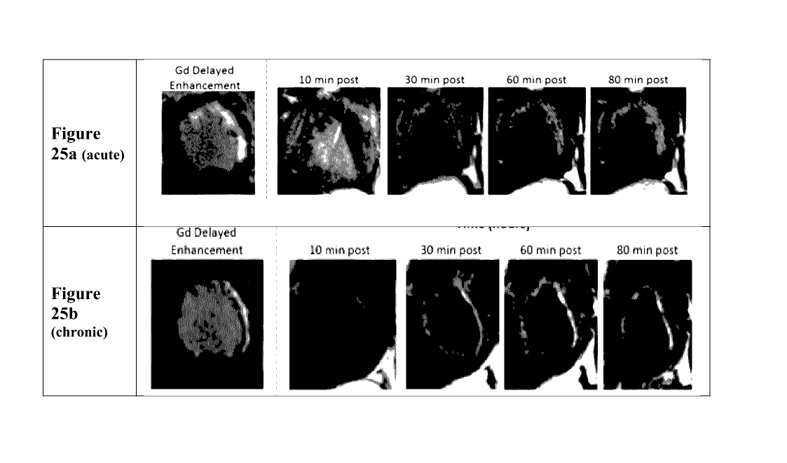

Prior to administration of Compound ID No. 1 the mean myocardial and infarct T1 values were 1192±22 ms and 1280±51 ms, respectively for acute MI, and 1182±37 ms and 1192±37 ms, respectively for chronic MI. Overall, Compound ID No. 1 contrast kinetics were different in the setting of acute versus chronic infarction (FIG. 24a and FIG. 24b). In animals with acute infarction, both normal and infarcted myocardium showed an initial drop in T1 (FIG. 24a), with normal myocardium showing a larger drop in T1 at 10 min after Compound ID No. 1 administration. Thereafter, the T1 of normal myocardium increased over the course of 90 minutes. In infarcted myocardium, the T1 dropped further at the 30 minute time point, and remained relatively stable for the duration of the experiment. Example images using the fixed-TI sequence set to null precontrast myocardium at successive time points after Compound ID No. 1 administration are shown in FIG. 25a (acute). At the initial time point, the infarct appeared hypointense compared to normal myocardium. At the 30 minute time point, the signal intensity of the infarcted and normal myocardium were similar. At later time points, infarcted myocardium appeared hyperintense. CNR was markedly improved between infarct and blood when compared to extracellular gadolinium (Compound ID No. 1 CNR: 19±7.4 v. Gd CNR: 6.3±3.4) despite the fact that the dose of Compound ID No. 1 (0.0075 mmol/kg) is 26-fold lower than that of extracellular gadolinium (0.2 mmol/kg).

Animals with chronic infarction exhibited different contrast agent kinetics. In animals with chronic infarction, both normal and infarcted myocardium showed an initial drop in T1 (FIG. 24b). However, in contrast to acute MI, chronic infarction exhibited lower T1 than normal myocardium throughout the entire experiment. FIG. 24b shows that chronic scar shows early and persistent lowering of T1 values, indicating increased uptake of Compound ID No. 1 compared to healthy myocardium.

Example images at successive time points after Compound ID No. 1 administration are shown in FIG. 25b (chronic). The chronic infarct showed enhancement at the initial time point, and became increasingly conspicuous at later time points. The extent of the enhanced area using Compound ID No. 1 matched that of Gd delayed enhancement. CNR between infarct and blood was improved for Compound ID No. 1 when compared to conventional extracellular gadolinium (Compound ID No. 1 CNR: 11±2 v. Gd CNR: 5±1.3).

Histopathologic Comparison

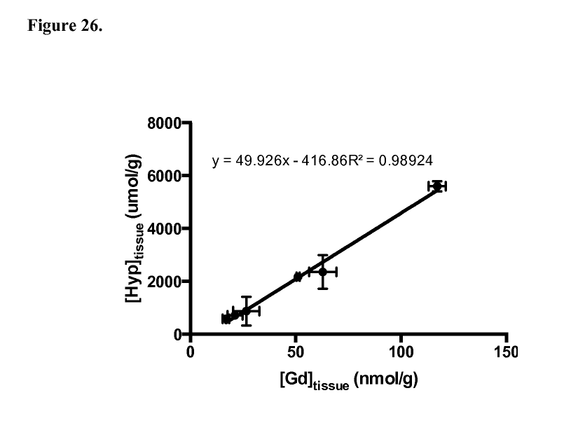

Masson Trichrome stain of tissue taken from infarcted and normal myocardium in both acute and chronic infarcts showed almost no staining for collagen (blue) within the acute infarct tissue, while chronic infarct showed dense fibrotic replacement of necrosis. The concentration of gadolinium (Compound ID No. 1) and hydroxyproline (collagen) was measured in samples of healthy, ischemic, and infarcted heart tissue (Table 11). The hydroxyproline concentration was slightly elevated in infarcted tissue (1,112±61.5 μg/g) compared to remote tissue (720±1.4 μg/g) for the animals with acute infarcts. Conversely, in the chronic infarcts which showed dense scar on Masson Trichrome stain, the hydroxyproline was strongly elevated in the infarcted tissue (3,973±2,294 μg/g) compared to remote tissue (793±113 μg/g). Importantly, there was a linear relationship of the concentration of Compound ID No. 1 vs. collagen concentration as measured by gadolinium (Compound ID No. 1) vs. hydroxyproline (collagen), FIG. 26. Since the MRI signal enhancement by the imaging probe Compound ID No. 1 is proportional to its concentration, these data show that Compound ID No. 1 enhanced imaging will enable quantitation of organ fibrosis.

TABLE 11
Concentration of gadolinium (Compound ID No. 1) and hydroxyproline
(collagen) in healthy, ischemic, and infarcted heart tissue.

| | |
|---|---|
| Gd nmol/g | Hyp (μg/g) |

| Animal | | Avg | Stdev | Avg | Stdev |
|---|---|---|---|---|---|
| #1 | Healthy | 21.23 | 3.59 | 711.97 | 154.92 |
| | Ischemic | 16.92 | 1.59 | 578.13 | 107.39 |
| | Infarct | 62.87 | 6.47 | 2350.36 | 630.58 |
| #2 | Healthy | 26.62 | 6.28 | 872.52 | 550.74 |
| | Ischemic | 51.15 | 0.73 | 2168.59 | 20.18 |
| | Infarct | 117.20 | 4.06 | 5594.81 | 197.22 |

Uptake into Fibrotic Liver Tissue

Bile duct ligated (BDL) rats are selected to assess the uptake of the complexes in fibrotic liver tissue as compared to sham operated animals. In this model, the common bile duct is surgically tied off and the resultant cholestosis results in fibrosis around the bile ducts. Laparotomy is performed in Sprague-Dawley rats with double ligation of the common bile duct with a section between the two ligatures (2-3% isoflurane anesthesia). Laparotomy without ligation is also performed as a sham control and to verify that the operation does not alter hepatic function. Fibrosis is evident 15 days after ligation and increases with time up to 30 days after ligation, providing defined endpoints for imaging of moderate and severe fibrosis.

Studies are conducted by injecting Compound ID. No. 1 at a dose ˜7.5 μmol/kg. After 90 min, the animals are sacrificed and the 4 liver lobes and abdominal muscle are removed and the gadolinium concentration quantified by ICP-MS and collagen content quantified using hydroxyproline concentration. The data are consistent with uptake of Compound ID No. 1 into fibrotic-rich liver tissue.

A number of embodiments have been described. Nevertheless, it will be understood that various modifications may be made without departing from the spirit and scope of the invention. Accordingly, other embodiments are within the scope of the following claims

1. A compound (Compound ID No. 5) having the following structure: 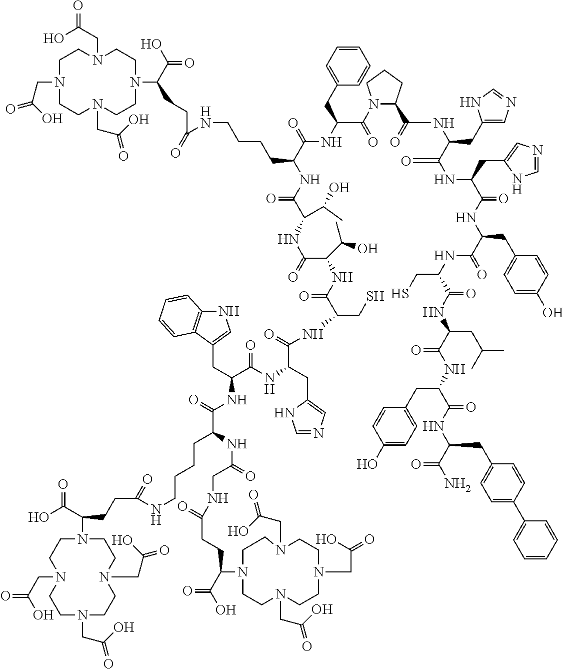
or a pharmaceutically acceptable salt thereof.

2. A compound (Compound ID No. 9) having the following structure: 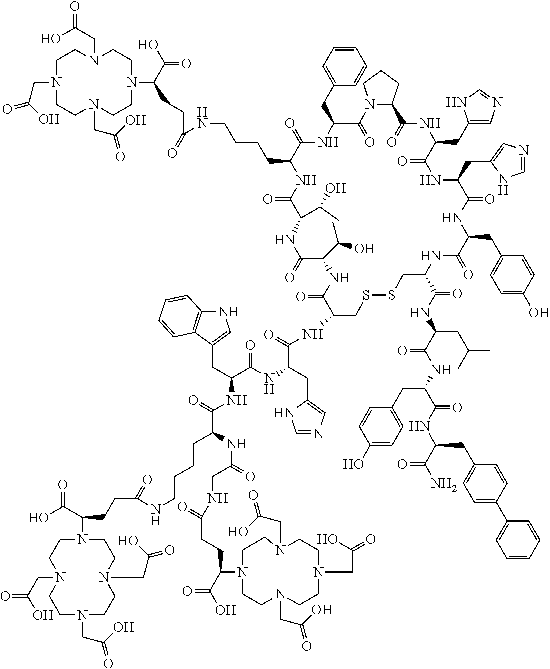
or a pharmaceutically acceptable salt thereof.

3. The compound of claim 1, wherein said compound is complexed to one or more paramagnetic metal ions.

4. The compound of claim 3, wherein said one or more paramagnetic metal ions is selected from the group consisting of: Gd(III), Fe(III), Mn(II), Mn(III), Cr(III), Cu(II), Dy(III), Ho(III), Er(III), Pr(III), Eu(II), Eu(III), Tb(III), and Tb(IV).

5. The compound of claim 4, wherein said paramagnetic metal ion is Gd(III).

6. The compound of claim 2, wherein said compound is complexed to one or more paramagnetic metal ions.

7. The compound of claim 6, wherein said one or more paramagnetic metal ions is selected from the group consisting of: Gd(III), Fe(III), Mn(II), Mn(III), Cr(III), Cu(II), Dy(III), Ho(III), Er(III), Pr(III), Eu(II), Eu(III), Tb(III), and Tb(IV).

8. The compound of claim 7, wherein said paramagnetic metal ion is Gd(III).

9. A compound (Compound ID No. 1) having the following structure: 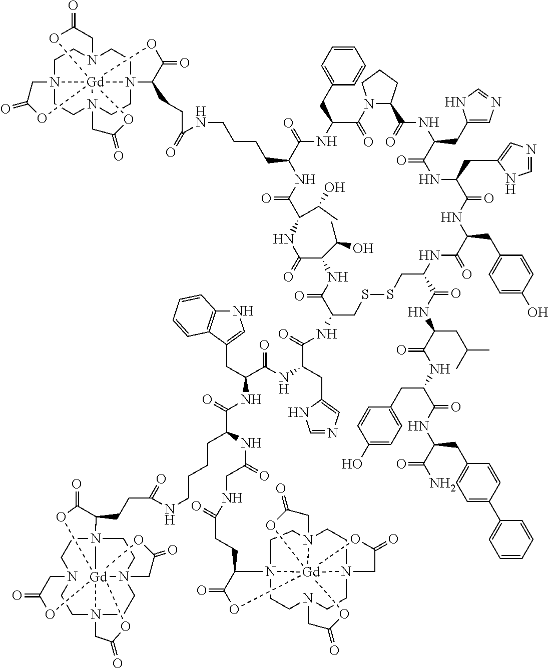
or a pharmaceutically acceptable salt thereof.

10. The compound of claim 6, wherein the compound is a sodium salt.

11. A method of distinguishing fibrotic from non-fibrotic pathologies in an animal, said method comprising:
a) acquiring a T1-weighted image of a tissue of said animal;
b) administering to the animal an effective amount of a magnetic resonance (MR) composition, the MR composition comprising a compound (Compound ID No. 1) having the formula: 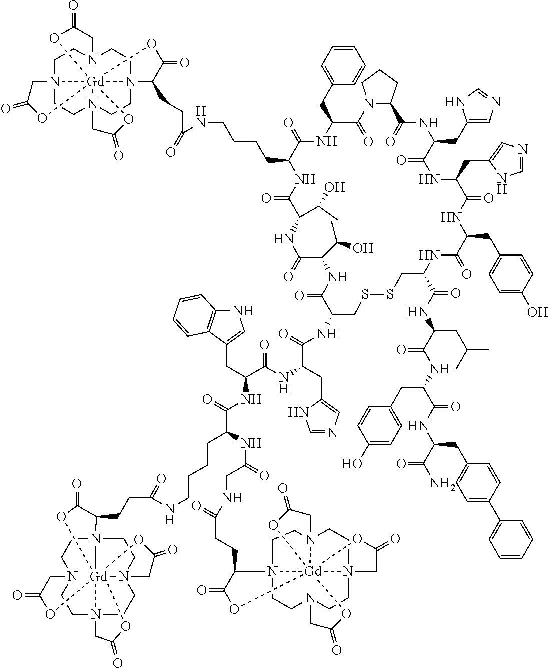
or a pharmaceutically acceptable salt thereof;
c) acquiring a T1-weighted image of a tissue of said animal at from about 1 minute to about 60 minutes after administration of the MR composition; and
d) evaluating differences between the images acquired in steps a) and c), wherein a fibrotic pathology exhibits greater signal increase in the image collected in step c) compared to the image in step a) as compared to non-fibrotic tissue.

12. A method of distinguishing fibrotic from non-fibrotic pathologies in an animal, said method comprising:
a) measuring R1 (1/T1) of a tissue of said animal;
b) administering to the animal an effective amount of a magnetic resonance (MR) composition, the MR composition comprising a compound (Compound ID No. 1) having the formula: 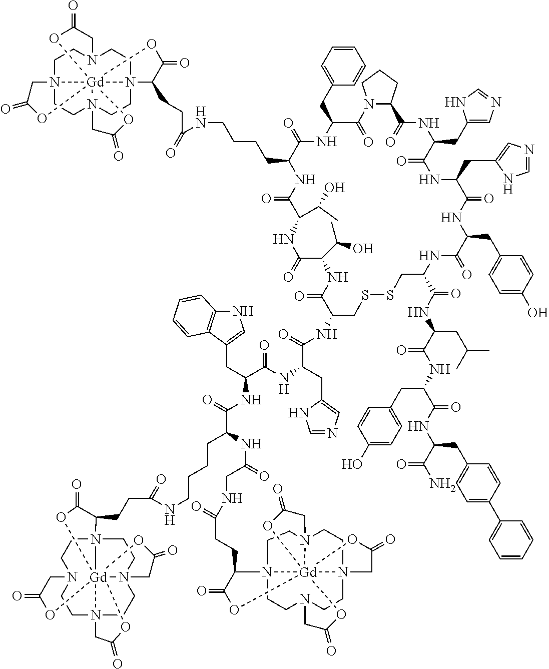
or a pharmaceutically acceptable salt thereof;
c) measuring R1 (1/T1) of a tissue of said animal at from about 1 minute to about 60 minutes after administration of the MR composition; and
d) comparing the difference in R1 of the tissue before and after administration of the MR composition comprising Compound ID No. 1 (delta-R1) to a reference value for that tissue whereby the tissue is fibrotic if the delta-R1value is greater than the reference value.


SEQUENCE LISTING (4 sequences)

SEQ ID NO: 1 - protein, 16 aa, Artificial Sequence
  synthetic peptide
GKWHCTTKFPHHYCLY

SEQ ID NO: 2 - protein, 15 aa, Artificial Sequence
  synthetic peptide
KWHCTTKFPHHYCLY

SEQ ID NO: 3 - protein, 16 aa, Artificial Sequence
  synthetic peptide
KYWHCTTKFPHHYCLY

SEQ ID NO: 4 - protein, 15 aa, Artificial Sequence
  synthetic peptide
KWHCYTKFPHHYCVY